(19) United States
(12) Patent Application Publication
(10) Pub. No.: US 2004/0033982 A1
(43) Pub. Date: Feb. 19, 2004

(54) Viral inhibition by n-docosanol

(76) Inventors: David H Katz, La Jolla, CA (US); Mohammed H Khalil, San Diego, CA (US); Laura E Pope, Carlsbad, CA (US); John F Marcelletti, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/312,321

(22) PCT Filed: Oct. 15, 2002
(86) PCT No.: PCT/US02/33019

(30) Foreign Application Priority Data
Oct. 16, 2001 (US) 60330444

(51) Int. Cl. A61K 31/7024

(52) U.S. Cl. 514/53

(57) ABSTRACT

This invention relates to topical therapeutic preparations and methods for treating viral and inflammatory diseases and for reducing the pain of topical inflammation of skin and mucous membranes. The preparations include creams containing n-docosanol.

### FIELD OF THE INVENTION

This invention relates to topical therapeutic preparations and methods for treating viral and inflammatory diseases and for reducing the pain of topical inflammation of skin and mucous membranes. The preparations include creams containing n-docosanol.

### BACKGROUND OF THE INVENTION

Most antiviral therapeutic compounds block various specific viral genetic replicative mechanisms within infected target cells. These approaches have drawbacks including toxicity to host cells, induction of drug-resistant viral sub-strains, and the potential to act as mutagens and/or teratogens for host cells. Consequently, the search for new antiviral compounds that provide efficacious therapy, without such deleterious consequences to the host, is of paramount importance.

Recurrent oral-facial herpes simplex (recurrent herpes simplex labialis, HSL) is a common disease estimated to occur in 20 to 40 percent of the United States' population. (Higgins C R, Schofield J K, Tatnall F M, Leigh I M J. Med. Virol. Suppl. 1:22-6, 1993). A main feature of the disease is the ability of herpes simplex virus (generally type 1 [HSV-1b)] to remain latent prior to erupting in response to such stimuli as stress; sunlight, fever, respiratory tract infections, and menstruation. (Spruance S L, in Clinical management of herpes viruses, Sacks S L, Straus S E, Whitley R J, Griffiths P D, editors, Amsterdam: IOS Press, p. 3-42, 1995). Episodes that do not progress beyond the papule have been referred to as aborted or nonlesional episodes. Classical lesions are those that progress to the vesiculo-ulcerative stage prior to healing.

HSL is self-limiting with healing normally occurring in 7 to 10 days. (Spruance S L, Overal J C, Kern E, Krueger G G, Pliam V, Miller W New Engl. J. Med. 297:69-75, 1997; Spruance S L Semin. in Dermatol. 11:200-6, 1992; and Shafran S D, Sacks S L, Aoki F Y, Tyrrell D L, Schlech W F 3^{rd}, Mendelson J, Rosenthal D, et al. J. Infect Dis. 176:78-83, 1997). Lesions evolve rapidly with maximum lesion severity often occurring within 8 hours of onset. (Spruance S L, Wenerstrom G. Oral Surg. 58:667-71, 1984). The window of time for therapeutic treatment is therefore small and it is essential that antiviral therapies be administered early. Antiviral therapies initiated at the papule or later stages cannot significantly affect lesion severity or the frequency of aborted lesions.

Compounds that exert antiviral activities without being potentially detrimental to the infected host have been identified and have shown some promising results. The oral antiviral medication valacyclovir hydrogen chloride is used to suppress genital herpes outbreaks, and for the treatment of recurrent outbreaks of genital herpes. In the late 1970's, for example, Snipes and colleagues (Snipes W, Person S, Keller G, Taylor W, Keith A Antimicrob. Agents Chemother. 11:98-104 (1977); Sands J, Auperin D, Snipes W Antimicrob. Agents Chemother. 15:67-73 (1979)) reported a series of studies demonstrating such activities for both saturated and unsaturated alcohols of moderate chain lengths. Optimal antiviral activity was observed with 10-12 carbon-long saturated alcohols; less antiviral activity was observed with alcohols 14-18 carbons long, and alcohols of higher chain lengths were not tested.

While significant antiviral activity was observed with C-10 and C-12 alcohols, these compounds also exhibited cytotoxic and hemolytic effects. Similar observations were made with unsaturated alcohols and monoglycerides, peak activity occurring with a C-18 alcohol containing three double bonds. Subsequently, Clark and colleagues (Clark L L, U.S. Pat. No. 4,670,471 (1987); McBride P T, Clark L L, Krueger G G J. Invest. Dermatol. 89:380-383 (1987)) concluded that the 30 carbon-long saturated alcohol, triacontanol, was active as an anti-herpes agent. However, since tissue culture studies demonstrated that triacontanol lacked direct antiviral activity, it was speculated that the apparent anti-herpes activity observed in animal studies might reflect an immunomodulatory effect of this compound.

As early as 1974, n-docosanol was reported to have systemic therapeutic value. For example, Debat, U.S. Pat. No. 4,186,211, reported that 1-docosanol when taken orally was therapeutically effective in the treatment of enlargement of the prostate gland. Similar work was reported a decade later by Yamamoto et al., e.g., U.S. Pat. No. 4,624,966, who, incorrectly as to chemical nomenclature, listed n-docosanol as a polyphenyl compound and described the peroral or parenteral administration of n-docosanol in therapy.

Compounds longer than 18 carbons have been examined to ascertain if they might exhibit topical antiviral or inflammatory activity (Katz et al., PCT Application No. WO 97/16434). Studies in our laboratory testing the antiviral properties of n-docosanol were favorable (Katz, D H, U.S. Pat. No. 4,874,794).

n-Docosanol inhibits in vitro a broad spectrum of lipid-enveloped viruses including HSV-1 and HSV-2, cytomegalovirus, varicella zoster virus, and human herpes virus 6. (Katz D H, Marcelletti J F, Khalil M H, Pope L E, Katz L R. Proc. Natl. Acad. Sci. USA 88:1082-9, 1991; Katz D H, Marcelletti J F, Pope L E, Khalil M H, Katz L R, McFadden R, Ann. NY Acad. Sci. 724:472-88, 1994; Marcelletti J F, Pope L E, Khalil M H, McFadden R R, Katz L R, Katz D H. Drugs of the Future 17:879-82, 1992; Pope L E, Marcelletti J F, Katz L R, Katz D H J. Lipid Res. 37:2167-78, 1996; and Pope L E, Marcelletfi J F, Katz L R, Lin J Y, Katz D H, Parish M L, Spear P G Antivir. Res. 40:85-94, 1998). Its mechanism of action is novel: following cellular incorporation and metabolic conversion, n-docosanol inhibits one or more steps of viral entry, blocking nuclear localization and subsequent replication of the virus. More recent experiments indicate that n-docosanol may exert anti-HSV activity predominantly by interfering with the process of viral fusion with the host cell. (Pope L E, Marcelletti J F, Katz L R, Lin J Y, Katz D H, Parish M L, Spear P G Antivir. Res. 40:85-94, 1998). in July 2000, n-docosanol 10 wt. % cream was approved by the U.S. Food and Drug Administration as an OTC topical treatment for recurrent oral-facial herpes simplex infections (trade name Abreva™).

The preparation of stable, efficacious n-docosanol-containing topical formulations presents a challenge. While creams and ointments of certain conventional formulations may be adequate for preliminary evaluations, certain excipients may be detrimental to the activity of n-docosanol. For example, penetration enhancers are often used as excipients in such formulations, but the effect on stabilizing activity of excipients in topical formulations may not be accurately predicted. Azone, reported by Rajadhyaksha, for example, is an excellent penetration enhancer but has not been known as a stabilizing constituent in cream formulations.

Sucrose esters of coconut fatty acids have been formulated as penetration enhancers, Cheng et al., U.S. Pat. No. 4,865,848, and other patents. Cheng et al., do not suggest, however, any cream stabilization resulting from these materials, nor is there any reason to infer such stabilization from the Cheng et al. patents. Literature on such compounds does not suggest these materials as being particularly effective in stabilizing C-20 to C-28 aliphatic alcohol-containing creams.

### SUMMARY OF THE INVENTION

The preparation of stable, efficacious n-docosanol-containing topical formulations presents a challenge. While creams and ointments of certain conventional formulations may be adequate for preliminary evaluations, certain excipients may be detrimental to the activity of n-docosanol. Therefore, there is a need for reproducibly effective formulations of n-docosanol that are stable for long periods of time, physiologically acceptable and suitable for topical application to skin and membranes.

In a first embodiment, a therapeutic cream is provided for application to skin and mucous membranes in the treatment of viral and inflammatory diseases including about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water.

In a second embodiment, a method of treating viral infections and inflammations of skin and mucous membranes is provided including applying to the skin or mucous membranes a stable therapeutic topical cream including about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water.

In a third embodiment, a method of reducing the pain of a surface inflammation of skin and mucous membranes is provided including applying to the inflamed surface a composition including about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water.

In a fourth embodiment, the use of a composition including about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water, in the preparation of a medicament for treatment of viral infections and inflammation of the skin or mucous membranes is provided.

In a fifth embodiment, the use of a composition including about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water, in the preparation of a medicament for reducing the pain of a surface inflammation of the skin or mucous membranes is provided.

In a sixth embodiment, a therapeutic cream is provided for application to skin and membranes in the treatment of viral and inflammatory diseases including sugar-based ester surfactant, greater than about 5 wt. % n-docosanol, mineral oil, an emollient co-solvent, and water.

In a first aspect of the sixth embodiment, the cream is stable at temperatures of at least 40° C. for a period of at least three months and after repeated freeze-thaw cycles.

In a second aspect of the sixth embodiment, the sugar-based ester surfactant is selected from the group consisting of sucrose cocoate, sucrose stearates, and sucrose distearate.

In a third aspect of the sixth embodiment, the sugar-based ester surfactant includes at least one compound selected from the group of sucrose esters consisting of sucrose cocoate, sucrose stearates and sucrose distearate, wherein sucrose ester (s) include about 3 wt. % or more of the cream. In another aspect the sucrose ester(s) include about 5 wt. % or more of the cream.

In a fourth aspect of the sixth embodiment, the emollient co-solvent is selected from the group consisting of polyoxypropylene stearyl ether, ethyl hexanediol, and benzyl alcohol, or combinations thereof.

In a fifth aspect of the sixth embodiment, the n-docosanol includes at least approximately 10 wt. % of the cream.

In a seventh embodiment, a stable, efficacious therapeutic cream is provided wherein a principal therapeutic composition consists essentially of n-docosanol, and wherein the cream base including one or more compounds selected from the group consisting of sucrose cocoate, sucrose stearates and sucrose distearate and one or more compounds selected from the group consisting of polyoxypropylene stearyl ether, ethyl hexanediol, and benzyl alcohol.

In a first aspect of the seventh embodiment, sucrose ester(s) include at least approximately 5 wt. % of the cream.

In a second aspect of the seventh embodiment, the n-docosanol includes at least approximately 10 wt. % of the cream.

In a third aspect of the seventh embodiment, the therapeutic cream has the formulation: n-docosanol making up from 5 to 15 wt. % of the total cream; sucrose stearates making up from 0 to 15 wt. % of the total cream; sucrose cocoate making up from 0 to 10 wt. % of the total cream; sucrose distearate making up from 0 to 10 wt. % of the total cream; with the proviso that at least one sucrose ester be present and make up at least about 3 wt. % of the total composition; mineral oil making up from 3 to 15 by weight of the total cream; benzyl alcohol making up from 0.5 to 10 wt. % of the total cream; and water making up from 40 to 70 wt. % of the total cream.

In an eighth embodiment, a method of treating viral infections and inflammations of skin and mucous membranes is provided including applying a stable therapeutic topical cream wherein the therapeutically active composition consists essentially of n-docosanol, and wherein the cream base consists essentially of sugar-based ester surfactant, at least one long chain aliphatic alcohol having from 20 to 28 carbon atoms selected from the group consisting of n-eicosanol, n-heneicosanol, n-tricosanol, n-tetracosanol, n-pentacosanol, n-hexacosanol, n-heptacosanol, and n-octacosanol, or mixtures thereof, mineral oil, an emollient co-solvent, and water.

In a first aspect of the eighth embodiment, n-docosanol includes more than one-half of the long chain aliphatic alcohols

In a ninth embodiment, a method of treating viral infections and inflammations of skin and mucous membranes is provided including applying a topical cream having the formulation: n-docosanol about 5-20 wt. %; sucrose stearates about 0-15 wt. %; sucrose cocoate about 0-10 wt. %; sucrose distearate about 0-10 wt. %, with the proviso that at least one sucrose ester be present and, wherein sucrose ester(s) include about 3 wt. % or more of the cream; mineral oil about 3-15 wt. %; propylene glycol about 2-10 wt. %; polyoxypropylene-15 stearyl ether about 0-5 wt. %; benzyl alcohol about 0.5-5 wt. %; with the proviso that either polyoxypropylene stearyl ether or benzyl alcohol be present in an amount of at least about 1 wt. %; and water about 40-70%.

In a first aspect of the ninth embodiment sucrose ester(s) include about 5 wt. % or more of the cream.

In a tenth embodiment, an anti-inflammatory and antiviral cream is provided having the formulation: n-docosanol about 5-20 wt. %; sucrose stearates about 0-15 wt. %; sucrose cocoate about 0-10 wt. %; sucrose distearate about 0-10 wt. %, with the proviso that at least one sucrose ester be present and wherein sucrose ester (s) include about 3 wt. % or more of the cream, mineral oil about 3-15 wt. %; propylene glycol about 2-10 wt. %; polyoxypropylene stearyl ether about 0-5 wt. %; benzyl alcohol 0-5 wt. %; with the proviso that either polyoxypropylene stearyl ether or benzyl alcohol be present in an amount of about 1 wt. % or more; and water about 40-70 wt. %.

In a first aspect of the tenth embodiment, sucrose ester(s) include about 5 wt. % or more of the cream.

In an eleventh embodiment, a method of reducing the pain of a surface inflammation of the skin or membrane is provided including applying to the inflamed surface a composition of n-docosanol in a physiologically compatible carrier, said n-docosanol including from about 5 to about 25 wt. % of said composition.

In a first aspect of the eleventh embodiment, the physiologically compatible carrier is a cream base that includes one or more compounds selected from the group consisting of sucrose cocoate, sucrose stearates and sucrose distearate and one or more compounds selected from the group consisting of polyoxypropylene stearyl ether, ethyl hexanediol, and benzyl alcohol.

### BRIEF DESCRIPTION OF THE DRAWINGS

FIGS. 1 through 3B and FIGS. 6A and 6B pertain to experiments involving herpes simplex virus type 1 (HSV-1), while FIGS. 4 and 5 and FIGS. 7 through 9 involve herpes simplex virus type 2 (HSV-2).

FIG. 1 presents the comparative activities of Formulation I (n-docosanol 10.0 wt. %; sucrose stearates 11.0 wt. %; sucrose cocoate 5.0 wt. %; mineral oil 8.0 wt. %; propylene glycol 5.0 wt. %; 2-ethyl-1,3-hexanediol 2.7 wt. % and purified water 58.3 wt. %), three different preparations of Formulation II (same as Formulation I except 5 wt. % sucrose stearates was replaced with sucrose distearate and ethyl hexanediol was replaced with an equivalent amount of polyoxypropylene-15-stearyl ether) and ZOVIRAX (acyclovir; Burroughs Wellcome Co., Research Triangle Park, NC; a treatment of HSV infections which inhibits activity of viral DNA polymerase) in inhibiting HSV-1-induced cutaneous lesions in hairless guinea pigs.

FIG. 2 presents the comparative activities of Formulation I, Formulation II, and Formulation IA (n-docosanol 10.0 wt. %; sucrose stearates 11.0 wt. %; sucrose cocoate 5.0 wt. %; mineral oil 8.0 wt. %; propylene glycol 5.0 wt. %; benzyl alcohol 2.7 wt. % and purified water 58.3 wt. %).

FIG. 3A shows a comparison of activities of Formulation I versus Formulation III (n-docosanol 10.0 wt. %; sucrose stearates 5.0 wt. %; mineral oil 8.0 wt. %; propylene glycol 5.0 wt. %; benzyl alcohol 2.7 wt. %; and purified water 58.3 wt. %).

FIG. 3B depicts data comparing the activities of certain modifications of these formulations in which the relative surfactant concentrations have been modified from that of Formulation I. Modifications of surfactant concentrations were found to have appreciable deleterious effects on the extent of drug activity.

FIG. 4 depicts data showing the dose-response relationship of Formulation III for the inhibition of HSV-2 induced cutaneous lesions in hairless guinea pigs.

FIG. 5 graphically represents data showing that n-docosanol containing cream based upon a sucrose ester surfactant system (Formulation III) also inhibits HSV-24induced cutaneous lesions in hairless guinea pigs.

FIG. 6A graphically depicts data that demonstrates that n-docosanol, formulated as a suspension using the surfactant Pluronic F-68, also inhibits HSV-1 induced vesicles when applied before vesicles are present. The suspension formulation did not contain any of the excipients of n-docosanol containing cream including benzyl alcohol.

FIG. 6B graphically depicts data that demonstrates that n-docosanol, formulated as a suspension in nonionic surfactant Pluronic F-68, also inhibits HSV-1 induced vesicles when applied after vesicles are present. The suspension formulation did not contain any of the excipients of n-docosanol containing cream including benzyl alcohol.

FIGS. 7 through 13 depict data elucidating the pharmacology of n-docosanol.

FIG. 7 depicts data showing that n-docosanol inhibits acyclovir-resistant HSV-2. Vero cells were cultured in 35-mm wells (6×10⁵ cells per well) in medium alone (=none) or in the presence of the indicated concentration of acyclovir, n-docosanol-Pluronic F-68 suspension or control suspension (Pluronic F-68 only). The cultures were inoculated 24 hours later with 150 PFU of either wild-type HSV-2 or an acyclovir-resistant laboratory isolate from the wild-type HSV-2 that was plaque purified and passaged in 20 μg/ml acyclovir 44 hours later, the plates were incubated, fixed, stained, and scored for numbers of plaques. The data presented are means of plaques scored from duplicate cultures. The percent inhibition observed in cultures treated with acyclovir or n-docosanol relative to untreated control cultures is denoted in parentheses.

FIG. 8 depicts data showing the dose response of the topical emulsion formulation of n-docosanol on cutaneous HSV in guinea pigs. The backs of hairless guinea pigs were cleaned and inoculated with purified HSV-2 by puncture of the skin with a tattoo instrument. Two hours after virus inoculation, the inoculation sites were either untreated or treated with 100 μl of n-docosanol-containing cream or control vehicle; the sites were similarly treated 24, 30, 48, 52, and 56 hours after virus inoculation. Vesicle number per site was determined at the indicated time points. The data are expressed as means and standard errors of vesicle number derived from duplicate sites per determination. The numbers in parentheses depict percent inhibition of vesicle number at treated sites as compared to the untreated sites.

FIG. 9 depicts data showing that HSV-2 remains on the surface of n-docosanol treated Vero cells for prolonged times. Vero cells were cultured as described in the legend to FIG. 7 and incubated overnight. The cultures were then chilled to 4° C., inoculated with 100 PFU of HSV-2, and incubated 3 hours at 4° C. At time zero the cultures were washed with medium, inoculated with fresh medium (containing the indicated inhibitor) and incubated at 37° C. At each indicated time period, the cultures were washed with citrate buffer (pH 2.5) and reinoculated with fresh medium (lacking inhibitor). After a total of 44 hours incubation the cultures were stained and scored for HSV-2-induced plaques. The data are expressed as geometric means and standard errors derived from triplicate cultures per group.

FIG. 10 depicts data showing that radioactive metabolites of n-[¹⁴C]docosanol display the properties of phosphatidylcholine and phosphatidylethanolamine. A portion (0.5 ml) of the methanol eluate of the silica lipid fractionation was evaporated under nitrogen, resuspended in 20 μl chloroform:methanol (3:2; v:v) and spotted on a silica thin layer chromatography (TLC) sheet. After development with chloroform:methanol:acetic acid:water (60:50:1:4; v:v:v:v), the positions of standards were determined by staining with iodine vapors and the cpm per fraction determined by scintillation spectrometry after cutting the plastic-backed sheet into 5 mm strips.

FIG. 11 depicts data showing that n-[¹⁴C]-Docosanol is metabolized more by Vero cells than by MDBK cells. Vero or MDBK cells were plated as described. n-[¹⁴C]-docosanol was added to 6 mM (0.24 mM Tetronic 908) and the cultures were incubated 72 hours at 37° C./CO₂. Cells were extracted and analyzed on TLC with hexane:diethyl ether:acetic acid (20:30:1; v:vv) as the developing solvent. With this solvent system the polar phosphatides remain at the origin. The position of migration of n-[¹⁴C]-docosanol is indicated. Duplicate plates were treated with an identical suspension lacking the radioactive label, and the numbers of cells in these duplicate plates were determined by counting cells excluding trypan blue with a hemocytometer.

FIG. 12 depicts data showing that n-docosanol inhibits in vivo Friend virus induced leukemia and viremia. Adult BALB/c mice were injected intravenously with 75 spleen focus-forming units of FV. Treated groups were injected intravenously with the indicated doses of n-docosanol or Pluronic vehicle alone on the same day as virus inoculation and once daily for the next 3 days. After 10 days, half of the animals in each group were sacrificed and examined for leukemic foci in their spleens (panel A). The remaining mice were retained 10 more days and bled for viremia determinations (panel B). Viremia was measured using the X-C plaque assay. Briefly, primary fibroblast cultures were derived by digestion of 14-day BALB/c embryos with trypsin and culturing in DMEM plus 10% fetal calf serum. After 72 hours, the cells were transferred into 16-mm dishes (10⁵/well), pretreated with 5 μg/ml polybrene and then infected with 75 X-C plaque-forming units of Friend virus stock or dilution of test plasma. After incubation for 7 days, the cultures were irradiated and overlaid with X-C cells (3×10⁵/well). Three days later, the cultures were washed, stained, and scored for plaques of multinucleated giant cells. The data presented are geometric means and standard errors of splenic foci or X-C plaque-forming units derived from three animals per group.

FIG. 13 depicts data showing that n-docosanol inhibits in vitro replication of HIV-1 in cultures of PHA/IL-2-stimulated human peripheral blood mononuclear cells. Human peripheral blood mononuclear cells were cultured in medium containing 1 μg/ml PHA plus 5 units/ml IL-2 alone or also containing 100 μg/ml PFA, the indicated dosage of n-docosanol/Pluronic F68, or the amount of Pluronic F-68 control vehicle contained in the high dose of n-docosanol/Pluronic F-68. After overnight incubation, the cultures were inoculated with HIV-1 at a multiplicity of infection of 1 virion/cell. After 24 hours incubation at 37° C., the cultures were washed and inoculated with fresh medium containing PHA and IL-2, but lacking inhibitor. Replication of HIV-1 was determined 4 days later by quantitation of viral antigens by a p24-specific ELISA for HIV-1.

FIG. 14 illustrates the Kaplan-Meier distributions for time-to-healing for treatment of acute HSL using n-docosanol 10 wt. % cream. Time-to-healing was measured from initiation of treatment until the date and time of the clinic visit at which complete resolution of all local signs and symptoms was clinician determined.

FIG. 15 provides a graphical depiction of HSV-1 inhibition in hairless guinea pigs with PEG formulations.

FIG. 16 provides a graphical depiction of HSV-2 inhibition in hairless guinea pigs with PEG formulations.

FIG. 17 provides a graphical depiction of HSV-2 vesicle numbers in hairless guinea pigs.

FIG. 18 provides a graphical depiction of HSV-2 inhibition in Hartley guinea pigs.

FIG. 19 provides a graphical depiction of HSV-2 vesicle numbers in Hartley guinea pigs.

FIGS. 20a and 20b show the inhibition of HSV-1 increases when cells are incubated with n-docosanol before viral addition and this inhibitory effect has a half-life of approximately 3 h. (A) Vero cells were plated and incubated with 9 mM n-docosanol, the corresponding control vehicle or no addition for 0, 3, 6, or 24 h prior to the addition of HSV-1. The viral plaque assay was continued and the number of p.f.u. determined. The data are expressed as % inhibition compared to wells receiving no treatment. (B) Vero cells were plated, n-docosanol or the corresponding control vehicle was added and cells were incubated at 37° C. in 10% humidified CO₂. After 21, 24, 25, 26, and 27 h (6, 3, 2, 1, and 0 h before the addition of HSV-1), media containing drug was removed and the cells were washed with media. After a total of 27 h, HSV-1 was added to all wells. Two hours later virus-containing media was removed and replaced with fresh media lacking virus or drugs. The cultures were incubated and processed for determination of the number of HSV-induced plaques as in (A).

FIG. 21 shows the uptake of HSV-1(KOS)gL86 into HEp-2 cells when incubated in n-docosanol-treated cells. After attachment of HEp2 cells to culture wells, n-docosanol-vehicle, vehicle alone, or no agent (control) was added. Five to six hours after infection, the cells were processed, X-gal was added, and the absorbance at 600 nm was determined.

FIG. 22 provides a graph demonstrating that n-docosanol suspended with Tetronic 908 inhibits the entry of HSV-2 (333) into CHO-IEβ8 cells. CHO-IEβ8 cells were seeded into 24-well plates. After cell attachment, heparin, n-docosanol-vehicle, vehicle alone, or no agent (control) was added. Five to six hours after infection, the cells were processed, X-gal was added, and the absorbance at 600 nm was determined.

FIG. 23 provides a graphic depicting experimental results for n-Docosanol-treated NC-37 human B cells, the cells exhibiting decreased fusion with octadecyl rhodamine B chloride-labeled HSV-2. NC-37 human B cells were inoculated in the presence of 15 mM n-docosanol, the corresponding concentration of Tetronic 908 (0.1 mM) or without addition. Cells were harvested and R-18-labeled HSV-2 was added to aliquots in the presence of compounds at their original concentration. Following incubation at 37° C. for the times indicated, cells were fixed and fluorescence intensity determined by FACScan.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

10%) Docosanol Cream

To prepare a cream, n-docosanol (98% pure; M. Michel and Co., New York, N.Y.), a water-insoluble compound, is mixed at 80° C. with sucrose cocoate, sucrose stearates, sucrose distearate, mineral oil, propylene glycol and polyoxypropylene-15-stearyl ether. Water was added and mixed in to finish the cream.

A cream can also be formed by adding all the materials except n-docosanol to water to form the cream base and blending the n-docosanol into the cream base.

The following proportions were found to be generally suitable: n-Docosanol 5-25 wt. % (or n-docosanol in mixture with at least one other long chain aliphatic alcohol having from 20 to 28 carbon atoms, i.e., n-eicosanol, n-heneicosanol, n-tricosanol, n-tetracosanol, n-pentacosanol, n-hexacosanol, n-heptacosanol, and n-octacosanol); sucrose stearates 0-15 wt. %; sucrose cocoate 0-10 wt. %; sucrose distearate 0-10 wt. % (with the proviso that at least one sucrose ester be present and that sucrose ester(s) comprise about 3 wt. % or more, preferably about 10 wt. % of the total composition); mineral oil NF 3-15 wt. %; propylene glycol USP 2-10 wt. %; polyoxypropylene-15-stearyl ether 0-5 wt. %; benzyl alcohol NF 0-5 wt. % (with the proviso that either polyoxypropylene stearyl ether or benzyl alcohol be present in an amount of 2 wt. %); purified water 40-70 wt. %. However, in certain embodiments other proportions may be preferred.

The following proportions were found to be generally optimal: n-docosanol 5-10 wt. % (or n-Docosanol in mixture with at least one other long chain aliphatic alcohol having from 20 to 28 carbon atoms, i.e., n-eicosanol, n-heneicosanol, n-tricosanol, n-tetracosanol, n-pentacosanol, n-hexacosanol, n-heptacosanol, and n-octacosanol); sucrose stearates 6 wt. %; sucrose cocoate 5 wt. %; sucrose distearate 5 wt. % (with the proviso that at least one sucrose ester be present and that sucrose ester(s) comprise about 3 wt. % or more, preferably about 10 wt. % of the total composition); mineral oil NF 8 wt. %; propylene glycol USP 5 wt. %; polyoxypropylene-15-stearyl ether 2-3 wt. %; benzyl alcohol NF 2-3 wt. % (with the proviso that either polyoxypropylene stearyl ether or benzyl alcohol be present in an amount of 2 wt. %); purified water 55-60 wt. %. However, in certain embodiments other proportions may be preferred.

A formulation containing 2-ethyl-1,3hexanediol instead of polyoxypropylene stearyl ether or benzyl alcohol and sucrose esters was also found to be effective. However, a component other than 2-ethyl-1,3-hexanediol may be preferred in certain embodiments, for example, in compositions intended for repetitive topical application.

An n-docosanol composition of a preferred embodiment (FORMULATION I) is described in Table 1 below:

TABLE 1
n-DOCOSANOL FORMULATION I

| INGREDIENT | WT. % | FUNCTION/RATIONALE |
|---|---|---|
| n-Docosanol | 10.0 | Active drug substance |
| Sucrose Stearates | 11.0 | Emulsifier, Emollient |
| Sucrose Cocoate | 5.0 | Emulsifier, Emollient |
| Mineral Oil NF | 8.0 | Emollient |
| Propylene Glycol USP | 5.0 | Co-solvent, humectant, skin-feel |
| | | modifier, auxiliary |
| | | preservative |
| 2-Ethyl-1,3-hexanediol | 2.7 | Co-solvent, auxiliary |
| | | preservative |
| Purified water qs ad | 58.3 | Vehicle medium |

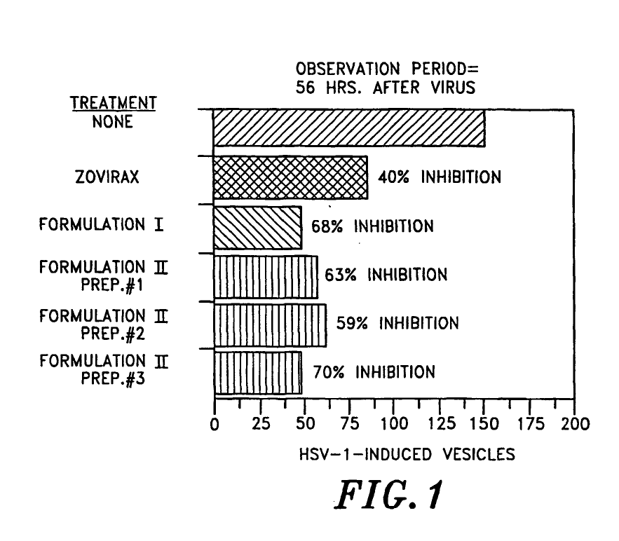

This n-docosanol cream was sufficiently stable for more than a short period of time to permit the carrying out of a comprehensive series of animal therapy trials in which the n-docosanol was found to be consistently active in the animal herpes model (FIGS. 1 through 3) and was used for the initial Phase I human clinical studies which showed it to be safe and tolerable. In certain countries outside of the United States, 2-ethyl-1,3-hexanediol may potentially be unacceptable for repetitive use. Therefore, in another embodiment it was preferred to substitute polyoxypropylene-15-stearyl ether for 2-ethyl-1,3-hexanediol, in equivalent amounts (2.7 wt. %), and 5 wt. % of the sucrose stearates were replaced with 5 wt. % sucrose distearate. The resulting n-docosanol composition (Formulation II) composition is described in Table 2, below:

TABLE 2
n-DOCOSANOL FORMULATION II

| INGREDIENT | WT. % | FUNCTION/RATIONALE |
|---|---|---|
| n-Docosanol | 10.0 | Active drug substance |
| Sucrose Stearates | 6.0 | Emulsifier, Emollient |
| Sucrose Cocoate | 5.0 | Emulsifier, Emollient |
| Sucrose Distearate | 5.0 | Emulsifier, Emollient |
| Mineral Oil NF | 8.0 | Emollient |
| Propylene Glycol USP | 5.0 | Co-solvent, humectant, skin-feel |
| | | modifier, auxiliary preservative |
| Polyoxypropylene-15 | 2.7 | Co-solvent, auxiliary preservative |
| Stearyl Ether | | |
| Purified water qs ad | 58.3 | Vehicle medium |

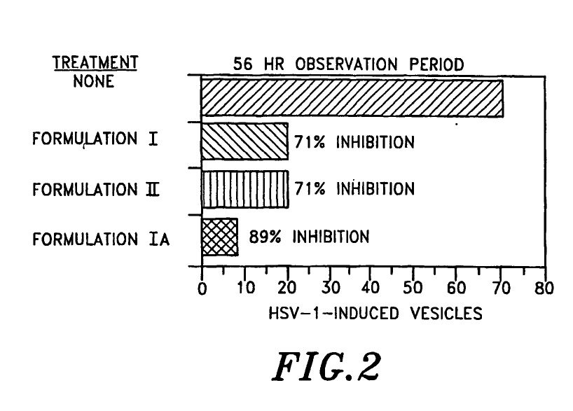

This modified Formulation II succeeded in providing physical stability to the final drug product and performed well in the guinea pig herpes animal model (see FIGS. 1 and 2). This formulation failed the USP preservative effectiveness test, however. Therefore, the formulation is only suitable for use in applications wherein passing the USP preservative effectiveness test is not necessary, i.e., certain non-human applications. Improved microbiological stability was achieved by replacing polyoxypropylene-15-stearyl ether with benzyl alcohol as co-solvent excipient, as described below.

In certain especially preferred embodiments providing stable compositions, only one or two surfactants of the classes described are used, wherein the surfactants are present in amounts of about 5 wt. %. The ability to use a limited number of types of surfactants and lower amounts of surfactant to produce stable creams was an unexpected and desirable result of our laboratory work. Excessive surfactant is not desirable because excess surfactant increases the potential for irritation at levels of surfactants above 5 wt. %. In addition, formulations with excessive amounts of nonionic surfactants frequently have problems with preservative effectiveness.

Utilizing several surfactant blends, with hydrophilic-lipophilic balance (HLB) values ranging from 9.0 to 13.0, a variety of n-docosanol creams were formulated and then screened for optimal emulsion quality, physical characteristics, drug efficacy and accelerated physical stability. Although most pharmaceutical emulsions are based on binary surfactant blends to optimize the HLB, test results revealed that sucrose stearates alone perform as well as or better than other surfactant blends in the improved n-docosanol formula. An n-docosanol formulation having such a surfactant blend (Formulation III) is as follows:

TABLE 3
n-DOCOSANOL (FORMULATION III)

| INGREDIENT | WT. % | FUNCTION/RATIONALE |
|---|---|---|
| n-Docosanol | 10.0 | Active drug substance |
| Sucrose Stearates | 5.0 | Emulsifier, Emollient |
| Mineral Oil NF | 8.0 | Emollient |
| Propylene Glycol USP | 5.0 | Co-solvent, humectant, skin-feel |
| | | modifier, auxiliary preservative |
| Benzyl Alcohol NF | 2.7 | Co-solvent, auxiliary preservative |
| Purified water qs ad | 69.3 | Vehicle medium |

The differences in Formulation III as compared with Formulation I include the replacement of 2-ethyl-1,3-hexanediol with benzyl alcohol, a well-known preservative and co-solvent with a long history of safe use and compendial status. The liquid nature and like functions of benzyl alcohol make it a rational and low risk replacement for ethyl hexanediol. The total surfactant level was reduced to 5 wt. % active with no change in the pharmaceutical characteristics of the product, no negative effect on the quality of emulsion based on microscopic examination, and no loss of physical stability in accelerated testing. Sucrose cocoate was omitted from the formulation without substantially affecting the properties of the formulation.

The cream can be made by heating and addition of ingredients, or by a more preferred method of combining oil-soluble ingredients and heating them separately from the water soluble components. The hot oil-soluble components are then added to the hot water phase while mixing vigorously. Table 4 summarizes certain evaluated formulations.

TABLE 4
FORMULATIONS (WT. % COMPOSITION)

| | | | | | FUNCTION/ |
|---|---|---|---|---|---|
| INGREDIENTS | I | II | IA | III | RATIONALE |
| n-Docasonol | 10.0 | 10.0 | 10.0 | 10.0 | Active Drug Substance |
| Sucrose Stearates | 11.0 | 6.0 | 11.0 | 5.0 | Emulsifier, emollient |
| Sucrose Cocoate | 5.0 | 5.0 | 5.0 | — | Emulsifier, emollient |
| Sucrose Distearate | — | 5.0 | — | — | Emulsifier, emollient |
| Mineral Oil NF | 8.0 | 8.0 | 8.0 | 8.0 | Emollient |
| Propylene Glycol | 5.0 | 5.0 | 5.0 | 5.0 | Co-solvent, auxiliary |
| | | | | | preservative |
| 2-Ethyl-1,3-hexanediol | 2.7 | — | — | — | Co-solvent, auxiliary |
| | | | | | preservative |
| Polyoxypropylene-15 stearyl ether | — | 2.7 | — | — | Co-solvent, auxiliary |
| | | | | | preservative |
| Benzyl Alcohol NF | — | — | 2.7 | 2.7 | Co-solvent, preservative |
| Water | 58.3 | 58.3 | 58.3 | 69.3 | Vehicle medium |

The n-docosanol Formulation III passed accelerated physical stability screening (storage at 42° C., freeze-thaw cycles) and also passed the USP preservative effectiveness test. Drug efficacy in the guinea pig herpes model was verified on repeated occasions.

To monitor stability, the n-docosanol cream formulations were stored, variously, at room temperature (30° C.), at elevated temperature (42° C.), and under freeze-thaw conditions in polypropylene jars. The freeze-thaw samples were subjected to 48 hours of freeze-thaw cycles, i.e., 24 hours at freezing temperature (−15° C.) and 24 hours at ambient room temperature. The cream samples, stored under the respective conditions, were visually inspected for physical stability at various time points. After 12 months at 30° C. or 3 months at 42° C. or 24 freeze-thaw cycles all samples remained as off-white creams. There was no evidence of syneresis or phase separation. Based on the above visual inspection, the Formulation III of 10 wt. % n-docosanol cream was considered to be physically stable when stored under any of the stated conditions.

The exact shelf life of Formulation III has not been determined but experience suggests that shelf life is more than adequate for a commercial n-docosanol containing cream. Thus, while certain n-docosanol formulations are unstable, specific formulations, Formulation III being preferred, have been found to be both stable and efficacious.

Those skilled in the art of formulating creams of hydrophobic and hydrophilic compounds will recognize that certain substitutions may be preferred in certain embodiments. Glycerol or another glycol may be preferred, with some adjustments in ratios, in place of propylene glycol, for example. Other polyoxyalkylene-based ethers may also be found to be substitutable for polyoxypropylene-15-stearyl ether. The relative proportions of the sugar-based esters may be varied considerably, so long as the total amount of sugar-based ester present is sufficient to stabilize the n-docosanol. This amount is preferably from about 5 to about 25 wt. %, although the minimum and maximum amounts have not been determined with precision.

In a particularly preferred embodiment, the formulation for n-docosanol cream is that of Formulation III containing 10 wt. % n-docosanol, 5 wt. % sucrose stearates, 8 wt. % mineral oil NF, 5 wt. % propylene glycol USP, 2.7 wt. % benzoyl alcohol NF and 69.3 wt. % purified water.

Long-term stable cream preparations that contain effective amounts of n-docosanol alone or in mixture with other such alcohols have been prepared, and the pharmacology of these compounds has been elucidated. In preferred embodiments, long-term stable topical creams formulation that have a shelf-life of greater than a year under normal handling conditions, i.e., is stable for a year or more at room temperatures and will withstand repeated freeze-thaw cycles, suitable for use in treating virus-induced and inflammatory diseases of the skin or membranes of an animal, including the treatment of humans, are provided. The ingredients of the cream include n-docosanol, alone or in mixture with other normal long chain (C-20 to C-28) aliphatic alcohols, as the physiologically active ingredient, water, oil, an ester of a sugar and a fatty acid, the ester being physiologically inert or capable of being metabolized by the body, and an emollient to assist in penetration of the n-docosanol into the affected area of the skin or membrane and co-act with the ester in forming a stable carrier for the physiologically active alcohol (s).

The sugar-based esters include a sugar moiety having a molecular weight of greater than about 150 and preferably above 250 and a fatty acid ester moiety having a molecular weight of about 150 or higher, and preferably above 250. The ester has a molecular weight of about 400 or higher. Sugars, as the term is used here, are sweet or sweetish carbohydrates that are ketonic or aldehydic derivatives of higher polyalcohols, and include both saccharides and disaccharides, disaccharide-based esters being preferred. High molecular weight polyhydric alcohols may be substituted for the more traditional sugars. Examples of such esterified sugar-based surfactants can be found in the chemical literature generally and in various catalogs, e.g., McCutcheon's directories, Volume 1-EMULSIFIERS & DETERGENTS, and Volume 2-FUNCTIONAL MATERIALS, (McCutcheon's Division, The Manufacturing Confectioner Publishing Co., Glen Rock, N.J., USA, 1993). Sucrose-fatty acid esters are preferred. Sucrose stearate and sucrose distearate are nonionic surfactants that are preferred for use in n-docosanol cream formulations to emulsify the oil and aqueous phases of the cream. These surfactants have a non-irritating nature, which makes them particularly preferred for treating, e.g., blisters caused by herpes virus. Sucrose stearates, when compared to conventional surfactants (such as surfactants marketed by ICI Americas of Wilmington, Del. under the tradenames Brij, Myrj, and Span) demonstrate superior properties as a surfactant for n-docosanol.

Propylene glycol is preferred for use in n-docosanol cream formulations as having a long history of safe use in topical formulations. One of the uses of propylene glycol in cream formulations is as a humectant to give a smooth supple feeling to the skin. Mineral oil is also preferred for use in n-docosanol cream formulations. Together with the n-docosanol, it forms the liquid phase of preferred cream formulations. Mineral oil has a long history of safe use in topical products and may perform such functions as acting as an emollient, e.g., by acting as a barrier to transdermal water loss, and to improve the texture of topical products.

Certain of the pharmacological studies were conducted using suspensions that are more compatible with the cells used in these studies but which are not suitable for use as topical pharmaceutical preparations in certain embodiments as they may lack the body and stability required for effective topical treatment.

A generally preferred cream formulation of certain embodiments includes, by weight based on the total weight of the final cream formulation, n-docosanol, typically about 5 to about 16, 17, 18, 19, 20, 21, 22, 23, 24, or 25 wt. %, more preferably about 6, 7, 8, or 9 wt. % to about 11, 12, 13, 14, or 15 wt. %, most preferably about 10.0%; sucrose stearates, typically about 0 to about 11, 12, 13, 14, or 15 wt. %, preferably about 1, 2, or 3 wt. % to about 4, 5, 6, 7, 8, 9, or 10 wt. %; and/or sucrose cocoate; typically about 0 to about 11, 12, 13, 14, or 15 wt. %, preferably about 1, 2, or 3 wt. % to about 4, 5, 6, 7, 8, 9, or 10 wt. %; and/or sucrose distearate typically about 0 to about 11, 12, 13, 14, or 15 wt. %, preferably about 1, 2, or 3 wt. % to 4, 5, 6, 7, 8, 9, or 10 wt. %; at least one sucrose ester or an equivalent sugar-based ester comprising typically at least about 3%, preferably about 4 wt. % to about 6, 7, 8, 9, 10, 11, 12, 13, 14, or 15 wt. %, most preferably about 5.0 wt. % of the total composition; oil, e.g., mineral oil NF typically about 3 wt. % to about 15 wt. %, preferably about 4, 5, 6, or 7 wt. % to about 9, 10, 11, or 12 wt. %, most preferably about 8.0 wt. %; a glycol, e.g., propylene glycol USP or equivalent, typically about 2 wt. % to about 8, 9, or 10 wt. %, preferably about 3 or 4 wt. % to about 6 or 7 wt. %, most preferably about 5.0 wt. %; an emollient glycol ether, e.g., polyoxypropylene-15-stearyl ether, or benzyl alcohol, typically about 0 to about 3.5, 4, 4.5, or 5 wt. %, preferably about 0.5, 0.75, 1, 1.24, 1.5, 1.75, 2, 2.25, 2.5, or 2.6 wt. % to about 2.75, 2.8, 2.9, or 3 wt. %, most preferably about 2.7 wt. %; and water typically about 40, 41, 42, 43, or 44 wt. % to about 70, 71, 72, 73, 74, 75,76, 77, 78, 79, or 80 wt. %, preferably about 45, 46, 47, 48, 49, 50, 51, 52, 53, 54, or 55 wt. % to 56, 57, 58, 59, 60, 61, 62, 63, 64, 65, 66, 67, 68, or 69 wt. %, most preferably about 69.3 wt. %. Within this general formulation, many specific formulations can be prepared which will be stable and which will exhibit the therapeutic effect noted based upon the data presented above, the teachings of the specification and the guidelines provided in the specification. Thus, an effective topical therapeutic composition wherein the therapeutically active material consists essentially of n-docosanol, alone or in mixture with normal long chain (C-20 to C-28) aliphatic alcohols may be prepared.

The formulations may be used in the manufacture of pharmaceuticals and also in the treatment of human and animal patients.

### EXPERIMENTS

To confirm in an experimental model the efficacy of n-docosanol cream on HSV-induced lesions, and to compare its activity to that of ZOVIRAX, hairless guinea pigs were inoculated with 1×10⁵ PFU of HSV-1, and then treated with either n-docosanol-containing or control cream, or ZOVIRAX ointment. The n-docosanol creams were constructed as described. The control cream was constructed in a similar manner except stearic acid was substituted for n-docosanol. Treatment was started either 2 or 48 hours after virus inoculation. The sites were evaluated for vesicle formation, defined as a pus-filled blister, at the indicated time points.

FIG. 1 presents the comparative activities of Formulation I and three different preparations of Formulation II as well as ZOVIRAX. Formulation I and Formulation II of n-docosanol creams both showed greater inhibitory power than ZOVIRAX ointment.

FIG. 2 presents the comparative activities of Formulation I, Formulation IA and Formulation II. Significant inhibition of HSV-1-induced lesions was demonstrated for all three formulations.

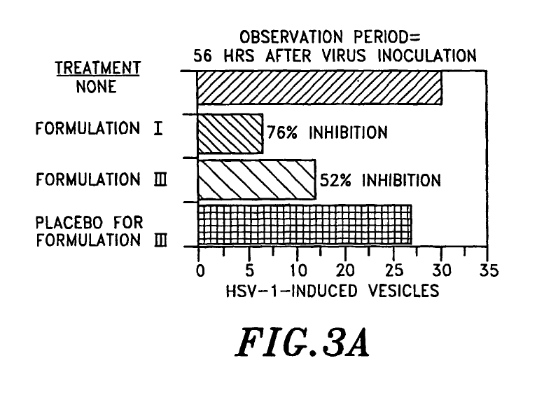

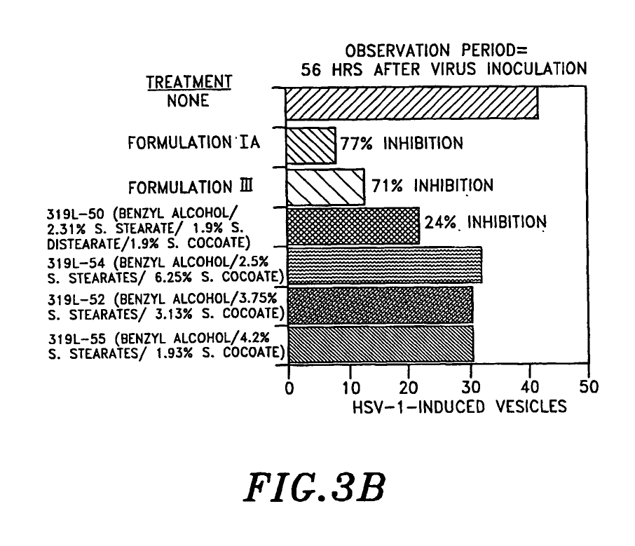

FIG. 3 shows a comparison of activities of Formulation III versus Formulation I and also depicts certain modifications of these formulations in which the relative surfactant concentrations have been modified from that of Formulation I.

Modifications of surfactant concentrations were found to have appreciable deleterious effects on the extent of drug activity. Formulation III was shown to have potent inhibitory power for HSV-I-induced lesions.

Volunteer patients with recurrent oral or genital HSV I or II infections have also been treated with topical n-docosanol-containing cream at various stages of an acute herpes outbreak. When treatment is initiated during the prodromal stage, n-docosanol cream may abort further progression of the infection (i.e., prevent vesicle formation). When treatment is started after vesicle formation has already occurred, n-docosanol cream may shorten the time for healing (i.e., complete re-epithelialization) of such herpes lesions.

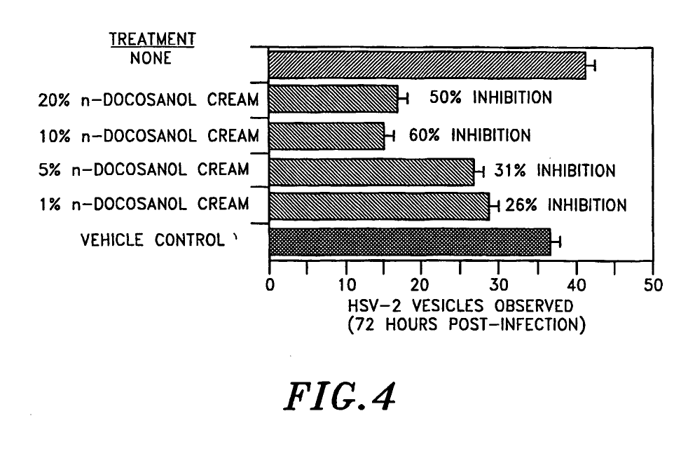

The selection of 10 wt. % n-docosanol in the formulation was tested in a dose-response study in the hairless guinea pigs. The sites on the backs of hairless guinea pigs were inoculated with HSV-2 as described previously. The sites were treated with 1, 5, 10, and 20 wt. % n-docosanol formulations. A vehicle control containing no n-docosanol was also included in the study. The results, illustrated in FIG. 4, show that after 72 hours of virus inoculation the untreated sites exhibited an average of 41 vesicles. Treatment with 20 wt. % and 10 wt. % n-docosanol containing cream inhibited vesicle number by 50% and 60%, respectively. Creams containing 1 wt. % and 5 wt. % n-docosanol were less effective than the 10 wt. % preparation. The control vehicle was without appreciable inhibitory effect.

It has been observed that the level of n-docosanol in the cream may play a role in the physical appearance, stability, and efficacy of n-docosanol cream. A comparison of creams containing 5, 10, and 20 wt. % n-docosanol was conducted. In general, it was observed that the viscosity of the product varied directly with the concentration of n-docosanol in the formulation (FIG. 4). The 5 wt °% formulation had the lowest viscosity with lotion-like appearance and had a tendency to separate into phases. The 20 wt. % formulation had the highest viscosity, was difficult to rub in and had a tendency to leave a white residue on human skin. Complete removal of n-docosanol from the cream resulted in a watery, lotion type formulation that underwent phase separation after overnight storage at room temperature. The 10 wt. % formulation was physically stable and cosmetically most pleasing, rubbing in easily and not leaving any residue on human skin. The results indicate that in addition to its function as an active ingredient, n-docosanol also functioned as a thickening agent and an emulsion stabilizer in the creams tested. In vivo studies with hairless guinea pigs showed that the 10 wt. % formulation had better efficacy than 5 wt. % or 20 wt. % formulations. While the 10% formulation was preferred for most applications, in certain embodiments, however, a formulation containing less than 10 wt. %, e.g., 5 wt. % or less n-docosanol may be preferred, while in other embodiments a formulation containing more than 10 wt. %, e.g., 20 wt. % or more n-docosanol may be preferred.

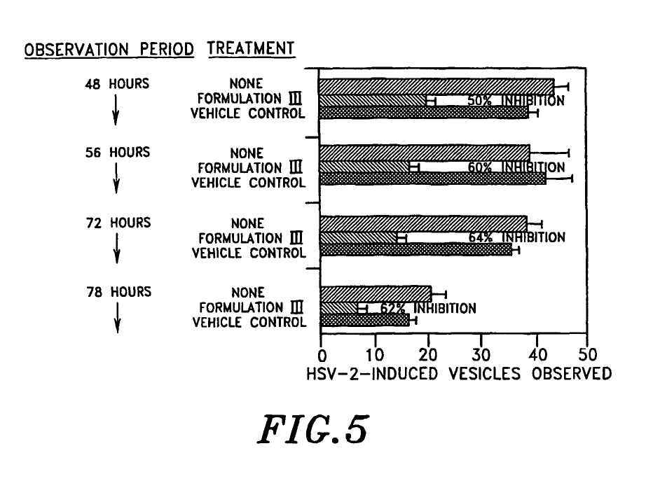

Since it has been reported that benzyl alcohol had some antiviral activity under certain circumstances, (Farah, A. E. et al., U.S. Pat. No. 4,200,655) a formulation of a preferred embodiment was tested to determine if benzyl alcohol acts as an antiviral reagent in the formulation. The cream containing benzyl alcohol and n-docosanol (10 wt. % n-docosanol cream) and the cream containing benzyl alcohol alone (placebo) were tested on HSV-2 induced cutaneous lesions in the hairless guinea pigs. Sites on the backs of guinea pigs were inoculated with HSV-2. The sites were treated as indicated in FIG. 5 and evaluated for vesicle formation at 48, 56, 72 and 78 hours after virus inoculation. There was an average of 44 vesicles in the untreated sites at the 48-hour time point, which remained relatively constant up to 72 hours after infection. At the 78-hour time point, resolution of the lesions became evident and by 96 hours post-inoculation vesicles were no longer visible. Treatment with n-docosanol cream inhibited vesicle number by 50-60% at the 48-56-hour time points, and by a slightly higher amount at the 72-78-hour points of analysis. Treatment with the control vehicle was without appreciable effect on vesicle number at any time point. Untreated and treated sites were excised and processed for viral culture. The presence of vesicles was directly correlated with the presence of infectious virus regardless of treatment or time of assay (not shown). Thus, vesicle number is an appropriate indicator for disease state in the studies described herein. Additionally, the cream and the placebo were tested in a phase II pilot study comprising sixty-eight patents with herpes labialis. The result of the double blind trial showed that early application of n-docosanol cream cut the duration of the episodes nearly in half. The treated groups' average outbreak period was 3.4 days, while the placebo group had outbreaks averaging 6.6 days.

The above results demonstrate that the presence of n-docosanol in the formulation is responsible for significant antiviral action.

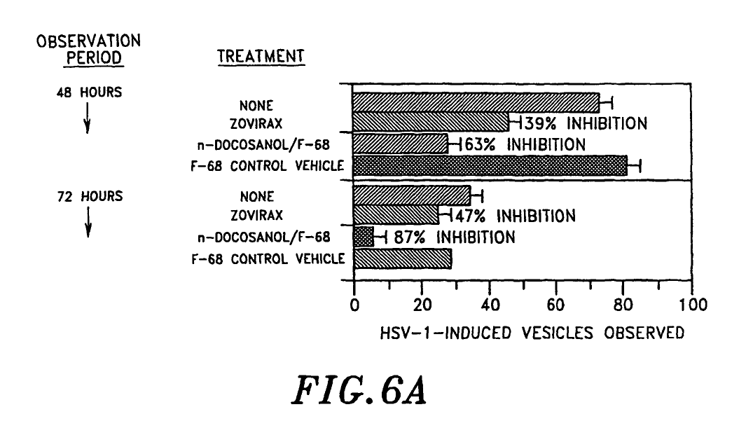

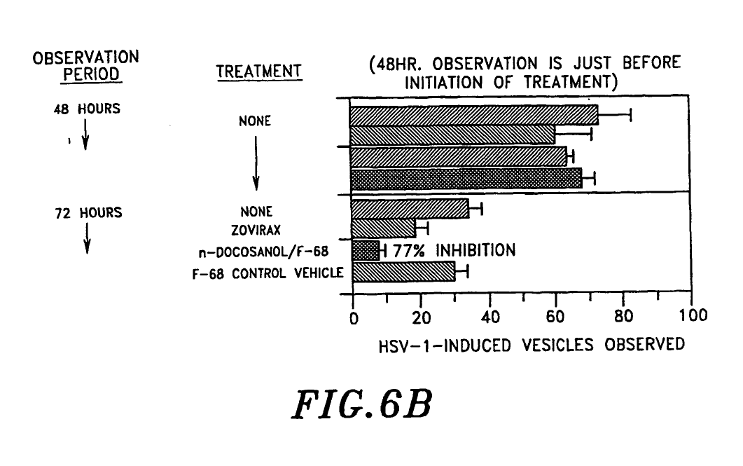

The antiviral activity of n-docosanol has also been demonstrated in a suspension formulation of n-docosanol in the nonionic surfactant Pluronic F-68 which did not contain any of the excipients of 10 wt. % n-docosanol cream formulation including benzyl alcohol. The results, summarized in FIG. 6, demonstrate two important points. First, as shown in panel A, a suspension formulation of n-docosanol in Pluronic F-68 also inhibits HSV-1 induced vesicles when applied 2 hours after virus infection, as observed with the cream formulation. Thus, the untreated sites exhibited an average of 74 vesicles at 48 hours after virus, but only 28 vesicles were observed in the sites treated with n-docosanol/Pluronic F-68 (63% inhibition). Treatment with ZOVIRAX, an FDA-approved treatment for certain HSV infections in humans, was also associated with decreased vesicle number, but less so than with n-docosanol.

Continued treatment with n-docosanol resulted in many fewer vesicles at the 72 hour time point also. The vehicle control for the n-docosanol preparation was without effect at either time point.

The second major point derived from FIG. 6 is that n-docosanol hastens resolution of HSV-1 induced disease even when administered after vesicles have emerged (Panel B). The various sites exhibited roughly equivalent number of vesicles at the 48-hour time point, which would be expected since none had been treated by that time. Vesicle numbers decreased in the untreated sites from a mean of 73 vesicles at 48 hours to 43 vesicles at 72 hours. Treatment with ZOVIRAX was associated with a modestly hastened disease resolution at 72 hours (27 vesicles, a 37% decrease versus the untreated sites), which is consistent with other experiments of a similar design. Importantly, application of n-docosanol/F-68 significantly hastened vesicle resolution as shown by the 77% inhibition of vesicle number when compared with the untreated group. The same conclusions were obtained using the cream formulation in experiments of a similar design. This demonstrates that n-docosanol need not be administered prophylactically to alter the HSV-induced course of disease.

Three safety and tolerance studies were conducted in healthy Caucasian male and female volunteers. A total of 78 healthy volunteers were exposed to drug. The safety studies indicated that the formulation of n-docosanol 10 wt. % cream does not cause phototoxicity, but is a mild primary irritant which also has the potential, albeit in low incidence, to cause allergic sensitization (1 subject of the 78 exposed experienced contact dermatitis).

Two clinical efficacy studies plus trials have been completed. Study A was a randomized, double-blind, placebo-controlled study in sixty-three patients (male and female) with recurrent herpes labialis. All of the thirty-one n-docosanol 10 wt. % cream-treated patients in the herpes labialis study, Study A, completed their treatment; two of those thirty-one patients reported a burning or stinging sensation after application of the cream.

No clinically significant changes in clinical laboratory values (blood chemistry, hematology, and urine analysis) were revealed in either study. Study B was a randomized, double blind, placebo-controlled trial in forty-four female patients with recurrent herpes genitalis. All of the twenty-two n-docosanol 10 wt. % cream-treated patients in the genital study, Study B, completed their treatment without reporting any drug-related adverse events.

Study A

Sixty-five patients (aged 16-60) took part in the Study A, thirty-two were initially randomized to receive 10 wt. % n-docosanol cream, and thirty-three were initially randomized to receive placebo cream. Treatment was patient-initiated, and treatment initiation was defined as “early” if the treatment started at prodrome or erythema stage and as “late” if started at the papule stage or later. Two patients were excluded from the analysis. Of the sixty-three evaluable patients, twenty-two were entered into the crossover phase of the study. In addition, thirteen patients treated more than one episode with the same study medication. Therefore, a total of ninety-eight herpes episodes—forty-eight treated with 10 wt. % n-docosanol cream and fifty treated with placebo cream—were analyzed.

The results of Study A are summarized according to first treatment episodes, crossover treatments and all treatment episodes combined in Table 5.

TABLE 5
STUDY A: TIME TO HEALING (DAYS) OF
RECURRENT HERPES LABIALIS EPISODES

| n-DOCOSANOL | | | | | |
|---|---|---|---|---|---|
| 10 WT. % | | | PLACEBO | | |
| MEAN | SD | (n) | MEAN | SD | (n) |

Part A. Analysis of first episodes

| | | | | | | |
|---|---|---|---|---|---|---|
| Early treatment | 2.5 | 2.4 | (10) | 6.8 | 4.2 | (4) |
| Late treatment | 6.8 | 3.2 | (21) | 7.3 | 2.7 | (29) |
| All treatments | 5.4 | 3.6 | (31) | 7.3 | 2.8 | (32) |

Part B. Analysis of crossover study

| | | | | | | |
|---|---|---|---|---|---|---|
| Early treatment | 2.7 | 2.2 | (7) | 7.0 | (1) | |
| Late treatment | 5.6 | 2.1 | (15) | 8.0 | 2.6 | (21) |
| All treatments | 4.7 | 2.5 | (22) | 8.0 | 2.5 | (22) |

Part C. Analysis of all treatment episodes in the study

| | | | | | | |
|---|---|---|---|---|---|---|
| Early treatment | 3.4 | 3.0 | (13) | 6.7 | 3.9 | (7) |
| Late treatment | 6.5 | 2.7 | (35) | 7.4 | 2.7 | (43) |
| All treatments | 5.7 | 3.1 | (48) | 7.3 | 2.9 | (50) |

Thirty-one patients treated their first episode of herpes labialis with 10 wt. % n-docosanol and thirty-two with placebo (Part A). Ten patients in the n-docosanol group and four in the placebo group were classified As early treatments. Mean healing time in the early-treatment n-docosanol group was 2.5 days, a reduction in mean healing time of 4.3-4.8 days compared with the other treatment modalities. This difference was statistically significant (P=0.0001) in favor of n-docosanol. In the late treatment cohort, n-docosanol reduced mean healing time in the first episodes by 0.5 day, which was not statistically significant.

Of the twenty-two patients entered into the cross-over study, the number who had treated their lesions early in both parts of the study (seven using n-docosanol in the crossover phase and one using placebo) was too small for meaningful statistical analysis (Part B). However, a substantial number (fifteen using n-docosanol in the crossover phase and twenty-one using placebo) had treated their lesions late, thus allowing for intra-patient comparison in this respect. Analysis of variance of the results of late treatment revealed a significant difference in favor of n-docosanol (P=0.03).

Evaluating the data from all ninety-eight treatment episodes of Study A together (single episodes, cross-over episodes and additional episodes with the same medication) reveals a statistically significant (P=0.02) reduction in mean overall healing time of 1.6 days in n-docosanol-treated (5.7 days) versus placebo-treated (7.3 days) patients (Part C). In the total twenty episodes classified as early treatments, topical n-docosanol reduced mean healing time by 3.3 days (P=0.05). Finally, when effectiveness of early treatment with n-docosanol was compared to all other treatment modalities, mean healing time in the early treatment n-docosanol group (3.4 days) differed quite significantly from the range of 6.5 to 7.4 days in the other groups; this difference was highly significant in favor of n-docosanol (P=0.0002). The differences between late treatment with n-docosanol 10 wt. % and early and late placebo treatment were not significant

As demonstrated by the data summarized in Table 5, early treatment with 10 wt. % n-docosanol cream (in the prodromal or erythema stage) produced a highly significant shortening of healing time compared with that obtained with the other treatments. In addition, late treatment, started after lesions had appeared, resulted in a statistically significant reduction in healing time in the n-docosanol-treated group in the crossover portion of the study, though not in the other analyses.

Study B

Sixty female patents with recurrent herpes genitalis entered the study while symptom-free and not in a prodromal stage. Thirty subjects were initially randomized to receive 10 wt. % n-docosanol cream and thirty to receive placebo cream in this patient-initiated trial for the treatment of early-stage herpes genitalis recurrences. Forty-four patients initiated treatment and returned to the clinic with a herpetic episode; twenty-two of these patients received n-docosanol and twenty-two received placebo.

The mean time to healing in the sixteen evaluable n-docosanol patients was 4.7 days ±1.9, ranging from 1.8 to 8.6 days; for the eighteen evaluable placebo patients, healing was complete within a mean of 5.1 days ±7.3, ranging from 1.7 to 10.4 days. The difference was not statistically significant (p=0.5827, t-test). Patients with non-genital lesions, who were noncompliant or had dosing interruptions, who had prodrome with no observable episode, or who had concurrent yeast infection, were considered nonevaluable. When all patients are included, the mean time to healing of the n-docosanol group was 5.5 days ±2.5, ranging from 1.8 to 9.8 days. For the placebo group, healing was achieved in a mean of 4.7 days ±2.3. Healing time in this group ranged from 1.7 to 10.4 days. There was no statistically significant difference in the mean time to healing between the two treatment groups (p=0.2703, t-test).

There was also no statistically significant difference between treatment groups when patients were stratified according to stage of the lesions (prodrome, erythema, or papule) when the treatment was initiated. The average healing time based on patient ratings was similar to the clinicians' (5.6 days for all n-docosanol patents versus 4.5 for all placebo patents).

Three pain analyses were conducted, based on patients' self-assessment of pain: time to sustained “no pain”; time to first “no pain”; and time to first reduction of pain.

Time to sustained “no pain” was measured from the time of first pain at application to the time when 1) pain was scored as “no pain” for a minimum of 2 consecutive recordings; and 2) during the remainder of the episode, additional pain recordings were no more frequent and severe than two separated episodes of two consecutive recordings of “mild” pain. Time to first “no pain” was defined as the interval from first pain at application to the first recording of “no pain.” Time to first reduction in pain was measured from the time of first pain at application to the first time when a decrease in pain level was noted, relative to the previous assessment. Several patents were excluded from these analyses because of either lack of pain within the first 24 hours, or noncompliance in reporting pain.

The fifteen evaluable patents treated with n-docosanol achieved a sustained response of “no pain” sooner than the fourteen evaluable placebo patient: a mean of 3.2 days ±1.9 for n-docosanol patients compared to 4.1 days ±2.5 for placebo patients. The n-docosanol patients also achieved “no pain” sooner than the placebo patients. The n-docosanol patents first recorded “no pain” a mean of 2.6 days ±2.1 after pain onset, while the placebo patients first reported “no pain” a mean of 3.4 days ±2.1 after pain onset. Among the evaluable n-docosanol patients, the first reduction in pain, relative to pain at the preceding application, occurred at a mean of 1.2 days ±1.0 after pain onset. First reduction in pain occurred in the placebo patients at a mean of 1.8 days ±1.4. These differences were not statistically significant (p=0.2775, 0.325, and 0.1757, respectively, t-test). Patients with non-genital lesions, who were noncompliant or had dosing interruptions, prodrome with no observable episode, and concurrent yeast infection, were considered nonevaluable.

In preferred embodiments, a method of reducing the pain of a surface inflammation of the skin or membrane including applying to the inflamed surface a composition of n-docosanol, optionally in combination with at least one long chain aliphatic alcohol having from 20 to 28 atoms selected from the group consisting of n-eicosanol, n-heneicosanol, n-tricosanol, n-tetracosanol, n-pentacosanol, n-hexacosanol, n-heptacosanol, and n-octacosanol, or mixtures thereof, in a physiologically compatible carrier, said alcohol including from about 5 to about 25 wt. % of said composition. Preferably, the physiologically compatible carrier is a cream base that includes one or more compounds selected from the group consisting of sucrose cocoate, sucrose stearates and sucrose distearate and one or more compounds selected from the group consisting of polyoxypropylene stearyl ether ethyl hexanediol and benzyl alcohol.

While no statistically significant differences were noted in Study B in time-to-healing between patients who received 10 wt. % n-docosanol cream and those who received placebo cream, a trend towards reduced time-to-healing among the evaluable patients treated with n-docosanol was observed. Three different pain analyses all showed a more rapid resolution of pain in the subjects who received n-docosanol 10 wt. % cream, though none of the differences were statistically significant. The inability to detect statistical significance in this study may reflect, in part, (1) the small study population; (2) differences at study entry between the two study groups with respect to the natural history of herpes genitalis lesions; and (3) an unequal distribution between the two groups of lesional stage at episode and treatment initiation.

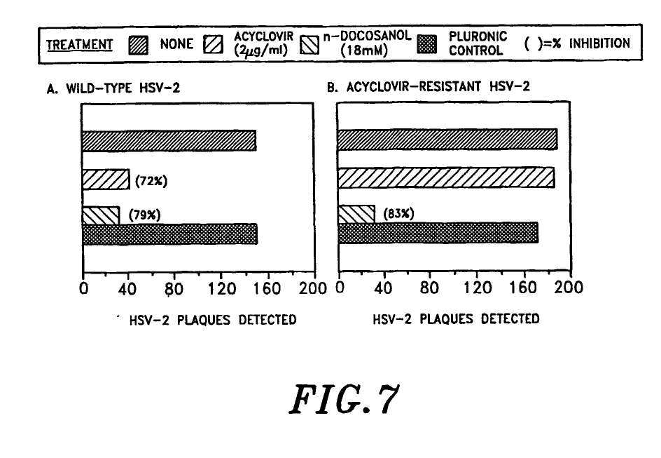

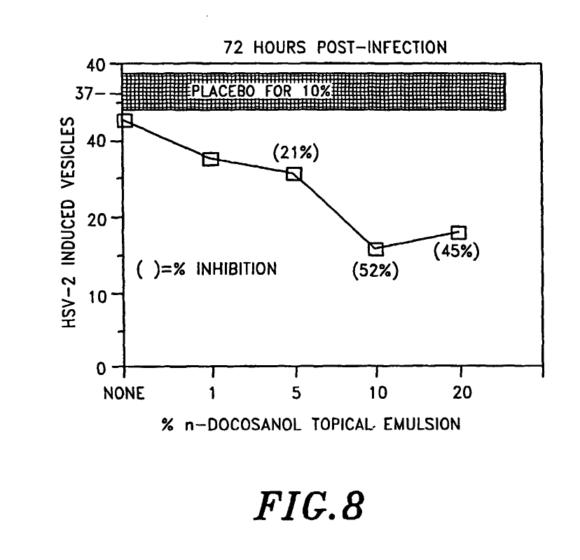

In addition to the clinical studies, several studies were carried out to elucidate the pharmacology of n-docosanol. These studies resulted in the data depicted in FIGS. 7 through 13, and are discussed below.

An appropriate formulation which allowed acceptable delivery of the n-docosanol to biological systems was developed. Initially, this was accomplished by formulating a suspension of the n-docosanol molecule in the inert and nontoxic nonionic surfactant, Pluronic F-68. Such suspensions are homogeneous, consisting of n-docosanol containing particles averaging 0.10 microns in diameter. Suspended in this way, n-docosanol exerts inhibitory activity in vitro against type 1 and 2 herpes simplex virus (HSV) infectivity of simian and human cell lines. n-Docosanol/Pluronic suspensions are equally effective against wild-type and acyclovir-resistant mutants of HSV.

Thus, as shown in FIG. 7, Panel A, acyclovir and n-docosanol inhibit plaque formation by wild type HSV-2 equally. FIG. 7, Panel B illustrates that an acyclovir-resistant HSV-2 mutant is not inhibited by acyclovir, but is inhibited by n-docosanol. The Pluronic surfactant alone lacks any antiviral activity. Host cell toxicity was not observed with n-docosanol at concentrations as high as 3 mM.

Extensive studies designed to delineate the mechanism by which n-docosanol exerts its antiviral activity were conducted. The collective implications of the results of the studies are that the compound appears to interfere with one or more of the common pathways of viral entry into the cell and migration to the nucleus of infected target cells. The key points of evidence supporting this notion can be summarized as follows: (a) the compound has no direct viricidal activity, since virus can be mixed with a n-docosanol suspension, then recovered from the suspension and shown to retain normal infectivity; (b) although the compound does not interfere with binding of herpes virus to HSV-specific receptors on target cells, HSV virions which have bound to target cell receptors in the presence of n-docosanol remain on the cell surface for a prolonged time period; and (c) subsequent migration to the cell nucleus of virus which has been internalized is inhibited, as measured by detectable HSV core and envelope protein, numbers of cells expressing the immediate early protein, ICP-4, and secondary plaque assays.

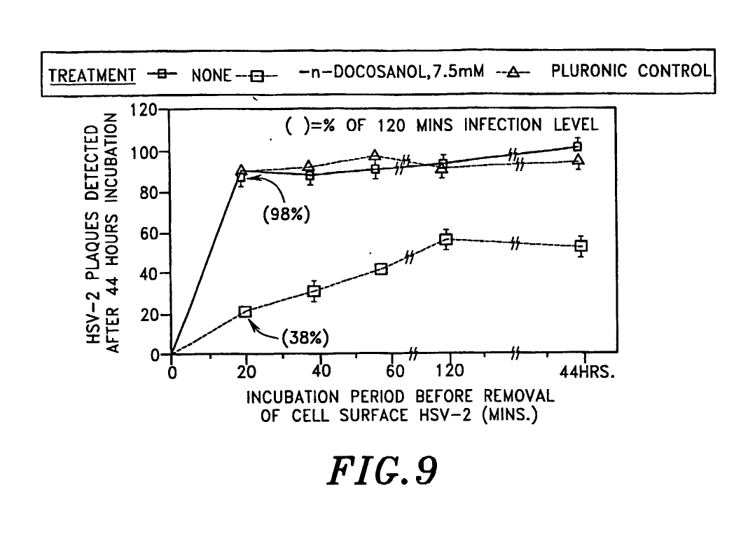

The delay in virus internalization described above is illustrated in the experiment summarized in FIG. 9. In this experiment, HSV-2 was incubated with Vero cells in the absence or presence of n-docosanol at 4° C. to allow for receptor binding of the virus. At the end of 3 hours, all cultures were washed and then replated at 37° C. in order to initiate the viral entry process. At 20 minute intervals thereafter, the various cultures were exposed to pH 3.0 citrate buffer, conditions which remove and inactivate surface-bound, but not internalized, HSV virions, and then re-cultured the full 44 hour period required to develop optimal HSV plaques. All cultures exposed to citrate buffer at time-0 failed to develop plaques, as expected.

As shown by the uppermost lines on the graph, internalization of HSV-2 is virtually complete within 20 minutes after the shift to 37° C. in the untreated and Pluronic control-treated cultures. In contrast, internalization of HSV in the n-docosanol treated cultures was less than 40% complete by 20 minutes and required more than 1 hour to reach completion. These results clearly indicate that the kinetics of viral fusion and/or transmembrane migration are delayed in some way by n-docosanol.

Even after internalization reaches completion in n-docosanol-treated cells, subsequent viral migration to the cell nucleus is significantly inhibited. Thus, the amounts of both HSV core and envelope protein antigens detectable by ELISA, as well as the numbers of infected cells expressing the intranuclear HSV-specific immediate-early protein, ICP4, by immunofluorescence, are reduced by more than 80%. Finally, the replication of infectious virions as measured in secondary plaque assay cultures is markedly diminished by 99% or more in n-docosanol treated cells.

To summarize, the presence of n-docosanol has no effect on the initial steps of viral binding, but considerably delays entry of virus into the target cell cytoplasm through some yet-to-be-determined mechanism. In addition, the process of migration to, and localization in, the nucleus is substantially blocked, having the ultimate effect of a marked decrease in productive viral replication.

In order to better define the precise mechanism by which n-docosanol exerts its antiviral activity, the cellular uptake, distribution, and metabolism of n-docosanol from surfactant-stabilized suspensions has been studied. The results of such studies have provided some interesting insight into the metabolic basis of the compound's antiviral action. It has been shown that radioactively labeled n-docosanol is progressively incorporated into cultured Vero cells, reaching a peak uptake per cell between 6 and 12 hours after exposure. The process is irreversible, since once the compound is cell-associated it cannot be removed even with extensive washing with cesium bromide, which effectively removes nonspecifically associated cell-bound particles.

Second, at saturating concentrations, less than 1% of the total n-docosanol added to cultures becomes cell-associated within 24 hours. Nonetheless, this corresponds to nearly 8×10⁹ molecules per cell, an amount which approximates the number of lipid molecules typically found in plasma membranes.

The fact that such a small fraction of n-docosanol in the suspension added to cultures becomes cell-associated indicates that the actual bioactive dose is orders of magnitude less than the amount of drug added to the cultures.

Cellular distribution studies examining subcellular fractions recovered by differential centrifugation of sonication disrupted cells demonstrated that after 12 hours of exposure 75% of the radioactive compound is contained in cell membranes, and less than 1% is associated with nuclear fractions; the balance of radioactivity is associated with the soluble cytoplasmic fraction.

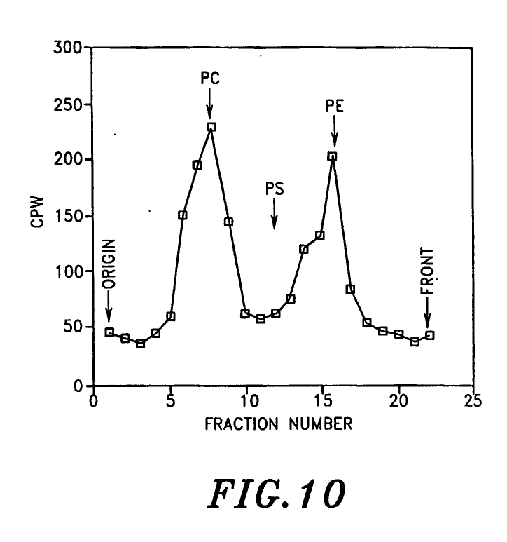

Analyses of the metabolic conversions of n-docosanol have shown that the compound is progressively metabolized to polar compounds, which were demonstrated by thin layer chromatography to be phosphatides, generated either via anabolic (ether linkages) or catabolic (oxidative) reactions. FIG. 10 demonstrates a thin layer chromatographic analysis of a methanol eluted (phosphatide-containing) fraction from a silica gel column of an extract of n-docosanol-treated Vero cells. Nonmetabolized n-docosanol was previously eluted from the silica with chloroform. As shown, approximately 62% of the counts migrated in the region of phosphatidylcholine and 38% migrated in the region of phosphatidylethanolamine.

Our studies have also documented that such metabolic conversions can be blocked by appropriate metabolic inhibitors. Thus, the effective energy poisons sodium azide and 2-deoxyglucose reduce both uptake of n-docosanol by Vero cells by 90% and metabolic conversion into polar metabolites by 80%. It is probable that the combination of sodium azide and 2-deoxyglucose mainly inhibits cellular uptake of n-docosanol by inhibiting endocytosis; however other mechanisms of uptake, including an energy-dependent fusion mechanism, or a passive diffusion mechanism facilitated by the subsequent energy-dependent metabolism of n-docosanol, could also be inhibited by these energy poisons.

An interesting aspect of these studies is the indication of a possible role for the polar metabolites of n-docosanol in the antiviral activity of the compound. It has recently been demonstrated that resistance of mouse fibroblasts to polyethylene glycol-induced fusion correlated with an increase in both free fatty alcohols and an elevation in glycerides, including an ether-linked compound that would be analogous to the products obtained via metabolic conversion of n-docosanol as described above.

Experiments were conducted to investigate the possibility that the enzymatic conversion of n-docosanol is a necessary prerequisite for its antiviral activity. The results of such studies have demonstrated, firstly, that the rate and extent of metabolic conversion, but not that of cellular uptake, of n-docosanol to its polar metabolites is determined by the nature of the surfactant used to suspend the compound and, indeed, that efficiency of metabolic conversion directly correlates with the magnitude of antiviral activity of n-docosanol.

An initial step in conducting such studies involved switching to a different surfactant or suspending n-docosanol. Tetronic 908 is closely related to Pluronic F68; both are block copolymers of ethylene oxide and propylene oxide. However, whereas Pluronic is a bifunctionaI polymer with a molecular weight of 8,400, Tetronic 908 is a tetrfunctional copolymer, produced by adding propylene oxide and ethylene oxide to ethylenediamine and resulting in a molecule with an average molecular weight of 25,000. Among other things, when Vero cells are exposed to equivalent doses of n-docosanol suspended in Tetronic versus Pluronic, the rate and extent of metabolism of the compound to polar metabolites is significantly higher with the Tetronic than the Pluronic suspension. The total uptake of radioactive n-docosanol was equivalent from the two different suspension formulations; only the metabolic conversion differed significantly. Correlating with this higher metabolic conversion from Tetronic than Pluronic suspensions is the finding that the ED₅₀ for inhibition of HSV replication by n-docosanol is 5-10 mM in Tetronic and approximately 3 times higher in Pluronic. This appears to relate to the 3-fold higher levels of metabolic conversion in cells treated with n-docosanol in Tetronic.

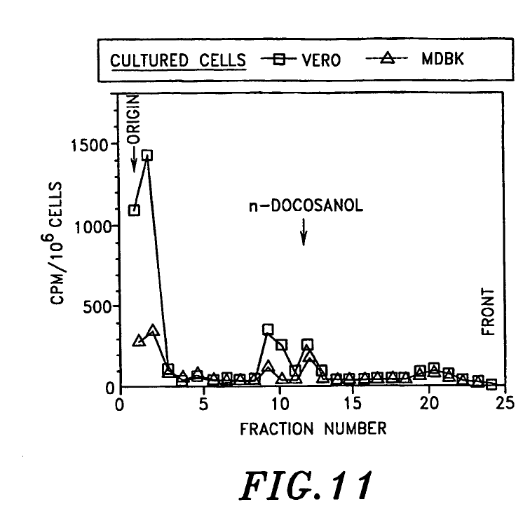

To eliminate the possibility that these findings are peculiar to the Vero cell culture system, a reciprocal analysis was made, taking advantage of the fact that, relative to Vero cells, the epithelial-like bovine kidney cell line, MDBK, exhibits an interesting apparent resistance to the anti-HSV activity of n-docosanol. This difference is significant in that n-docosanol is 3-4-fold more effective in inhibiting HSV-induced plaques in Vero cells than in MDBK cells. A comparison of total cellular uptake and relative metabolism showed that both the total amount of n-docosanol uptake and the relative amount of metabolic conversion were 3-4 times higher in Vero than in MDBK cells. The combined effect of decreased uptake and decreased metabolism in MDBK versus Vero cells is graphically illustrated in FIG. 11, which shows that after 72 hours, Vero cells contain almost 4-fold higher amounts of the phosphatide metabolite, which remains at the origin in this solvent system. Of the counts that are metabolized in two cells lines, the relative amounts in the major classes of phosphatides that are formed, phosphatidylcholine and phosphatidylethanolamine, are not different in the two cell lines. Moreover, pulse-chase experiments showed that both lines eventually convert all of the incorporated counts into the more polar form.

Such results suggest that MDBK cells may effectively regulate uptake and/or metabolism of n-docosanol through a feedback type mechanism that is either less effective or nonoperative in Vero cells.

Consistent with the mechanistic observations summarized above, it was predicted that n-docosanol would have potential for interfering with a variety of different viruses, specifically those which contain lipid in their outer envelopes and which use fusion mechanisms for entering susceptible target cells. Table 6 summarizes the human and murine lipid-enveloped viruses that have been shown to be susceptible to the antiviral activity of n-docosanol.

TABLE 6
SPECTRUM OF ANTIVIRAL ACTIVITY
OF n-DOCOSANOL AGAINST
LIPID-ENVELOPED VIRUSES

| HUMAN VIRUSES | MURINE VIRUSES |
|---|---|
| Herpes Simplex-1 & 2 | Cytomegalovirus |
| Varicella Zoster Virus | Friend Leukemic Virus |
| Human Herpesvirus-6 | LP-BM5 Virus |
| Respiratory Syncytial Virus | |
| Cytomegalovirus | |
| Influenza A | |
| HIV-1 | |

Every lipid-enveloped virus tested can be effectively blocked by this drug. n-Docosanol has anti-retroviral activity both in vito and in vivo. A formulation possessing anti-retroviral activity and lacking toxicity has substantial usefulness in treating a variety of retroviral diseases in humans and domestic animals. Notwithstanding the implications for treatment of AIDS, availability of a treatment regimen for diseases caused by retroviruses like feline leukemia virus, bovine leukemia virus, as well HTLV-1 and-2 has substantial benefits in humanitarian terms. Studies have established that n-docosanol inhibits replication of murine retroviruses in vitro and in vivo.

Initial studies focused on the murine Friend leukemia virus (FV; 8). Inoculation of adult mice with FV results in the induction of a leukemia of erythroid progenitors, specifically the basophilic erythmblast. This erythroleukemia is characterized by the rapid proliferation of virus-infected erythroid cells, viremia, immunosuppression, and ultimately death of the animal. Intravenously injected FV will circulate through hematopoletic organs, such as the spleen, and infect erythroid cells. If such infected spleens are fixed on day 10 after virus injection, discrete macroscopic nodules can be seen on the surface of the organ; these represent clones of leukemic cells and form the basis of the spleen focus assay.

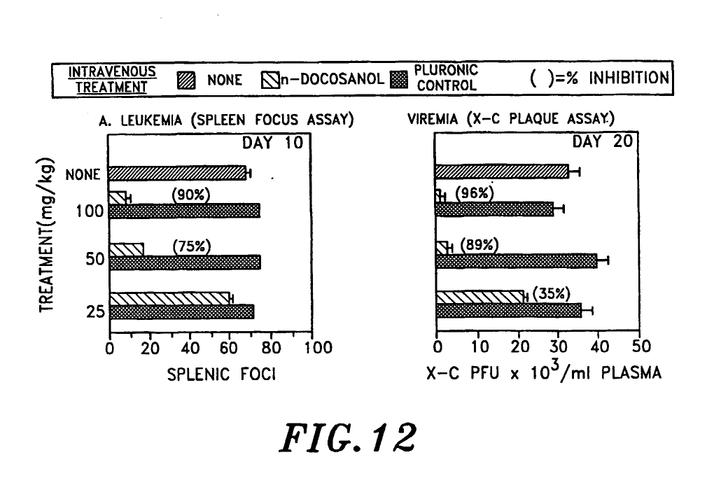

The experiment summarized in FIG. 12 illustrates that n-docosanol inhibits Friend Virus-induced leukemia add viremia injected intravenously with 75 focus-forming units of Friend Virus. Treated groups were injected intravenously with the varying doses of n-docosanol or Pluronic F-68 vehicle alone intravenously on the same day as virus inoculation and once daily for the next 3 days. After 10 days, half of the animals in each group were sacrificed and examined for the presence of leukemic foci in their spleens, while the remaining animals were retained for 10 additional days to monitor viremia. Treatment with n-docosanol exerted a very clear dose-related inhibitory affect on both the development of leukemic foci, shown in Panel A, and the development of viremia, shown in Panel B. In contrast treatment with comparable amounts of the Pluronic F-68 vehicle alone as control exerted no discernible effect. It is believed that these results reflect the inhibitory activity of n-docosanol on viral replication, since corollary in vitro studies have documented a very potent activity of this drug against replication of Friend Virus in primary embryo fibroblast cultures. n-Docosanol inhibits in vitro replication of HIV-1 and human herpes virus 6.

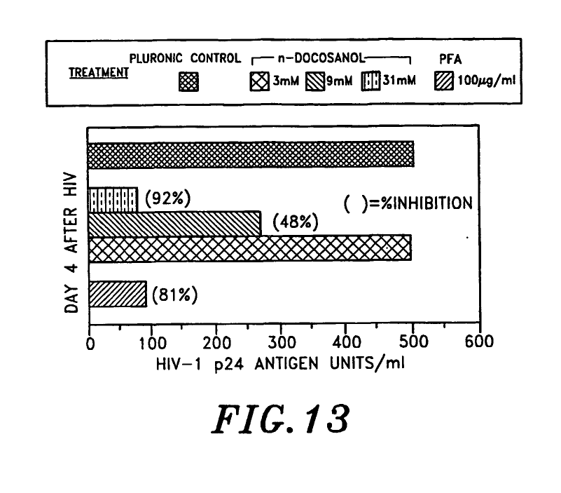

The initial studies on HIV were conducted in collaboration with a U.S. National Institutes of Health Laboratory and one of several experiments of this type is summarized in FIG. 13. Normal human peripheral blood mononuclear cells were activated with 1 μg/ml PHA plus 5 units/ml of IL-2 in medium alone or in the presence of n-docosanol, Pluronic F-48 control vehicle, or phosphonformic acid (PFA). The next day, the cultures were inoculated with HIV-1 and examined 4 days later for evidence of viral replication by detection of the p24 viral antigen. Substantial levels of HIV-1 replication occurred in the control-treated cultures, comparable to that observed in the untreated group. As shown, n-docosanol exhibited a dose-related inhibitory activity against HIV-1 in cultures of PHA/IL-2-stimulated human peripheral blood mononuclear cells. Activity at the highest dose was comparable to that observed with the very potent antiviral compound, phosphonoformic acid (PFA).

To determine whether n-docosanol 10 wt. % cream (docosanol) was efficacious compared to placebo for the topical treatment of episodes of acute HSL, two identical clinic-initiated, double-blind, placebo-controlled studies were conducted at a total of 21 sites. Otherwise healthy adults, with documented histories of HSL, were randomized to n-docosanol or polyethylene glycol placebo and initiated therapy in the prodrome or erythema stage of an episode. Treatment was 5 times daily until healing occurred (the crust fell off spontaneously or there was no longer evidence of an active lesion) with twice daily visits.

Each gram of n-docosanol 10 wt. % cream contained 100 mg n-docosanol formulated into a white, non-greasy, moisturizing cream that was easily applied and readily disappeared into skin and mucous membranes. The composition included n-docosanol 10.0 wt. %, sucrose stearate and sucrose distearate 5 wt. %, light mineral oil NF 8.0 wt. %, propylene glycol USP 5.0 wt. %, benzyl alcohol NF 2.7 wt. %, and purified water USP 69.3 wt. %. The composition is marketed under license from Avanir Pharmaceuticals under the tradename ABREVA™ by GlaxoSmithKline of Research Triangle Park, NC. A placebo formulation lacking n-docosanol but containing PEG provided a medication similar in appearance to n-docosanol 10 wt. % cream. The PEG formulation was identical to that utilized previously as a vehicle for topical acyclovir and as a placebo for topical HSL trials and was chosen in consultation with FDA. (See Spruance S L, Wenerstrom G. Oral Surg. 58:667-71, 1984; Spruance S L, Schipper L E, Overall J C, et al. J. Infect Dis. 146:85-90, 1982; and Fiddian A P, Ivanyi L. Brit. J. Dermatol. 109:321-6, 1983). In this instance it was not possible to use the vehicle of the cream as placebo, because the active drug substance, n-docosanol at a 10 wt. % concentration, is a major contributor to the consistency of the cream. Removing it produces a watery vehicle clearly unsuitable as a control for a blinded study.

Patients were recruited at twenty one sites including university clinics, private practices, and public health facilities across the U.S. Eight sites were assigned to study #06 and thirteen sites were assigned to study #07. All sites were included in the combined study, designated #06/07. No single site enrolled more than twelve percent of the total study population in the combined study or more than twenty-four percent in the individual studies. These sites recruited male and female immunocompetent patients 18 years of age or older who presented for clinical assessment within 12 hours of noticing the onset of prodrome or erythema. By patient history, signs and symptoms must not have been present for mote than 12 hours, and on clinical examination, the episode must not have progressed beyond the erythema stage. Patients, determined to be healthy otherwise, must have had a clinical history of HSL with at least two recurrences during the past 12 months. The most recent previous episode must have healed at least 14 days prior to screening. Institutional Review Board approval for all sites was obtained for the protocol and the informed consent document. All patients were properly informed of the study purpose and risks and a signed consent form was obtained prior to their enrollment.

Subjects agreed not to use cosmetics on or around the mouth during the treatment period. Women of childbearing potential were to be practicing an established method of birth control and were not to be pregnant as determined by a negative urine test at enrollment Subjects with known allergies to topical cosmetics were excluded as were those with lesions above the nares, below the chin, or inside the mouth. The use of any investigational drug during or within 30 days prior to the study and the use of an approved antiviral agent, topical corticosteroid, or any other non-specific therapy for HSL during or within seven days prior to the study were not allowed. Concomitant use of systemic corticosteroids or other drugs known to induce immune stimulation or immune suppression was also not allowed.

The study was a multicenter, randomized, double-blind, placebo-controlled, parallel group, clinic-initiated, early-treatment study to compare and evaluate the safety, efficacy, and tolerance of topical n-docosanol with a placebo in a population of patients with acute recurrences of HSL. Treatment was initiated within 12 hours of episode onset with symptoms in the prodrome or erythema stage and prior to the papule stage. Subjects were randomized in a double-blind fashion by site in blocks of four to receive either n-docosanol or placebo treatment. At study entry, the first application of study medication was to be made by the subject at the clinic. Subsequent applications were to be made by the subject during normal waking hours. Study medication was to be applied to the lesion area five times per day until healing for a maximum of 10 days. Subjects were instructed to re-apply study medication after heavy exercise, showering, or bathing. These extra applications were not counted as scheduled. Subjects kept a daily diary of study medication application times.

Subjects were required to report for twice daily assessments by the investigator or other trained clinician for the first seven days. Clinic visits could not be closer together than 6 hours or longer apart than 16 hours. The initial treatment area was marked on a diagram in the case report form (CRF) at the baseline clinical assessment. Localized signs and symptoms at the treatment area were documented at each visit, including prodrome/erythema, papule, vesicle, ulcer, crust, or healed skin (with or without residual erythema), and subject reports of pain, burning, itching, or tingling. Subjects with HSL episodes that did not abort or heal within seven days were also followed once per day for Days 8 to 10. HSL episodes that did not abort or heal within 10 days discontinued treatment and were again assessed at the point of lesion abortion, healing, or adverse experience. All baseline and efficacy and safety parameters were clinician-determined.

The primary efficacy endpoint (time-to-healing) was calculated from the date and time of therapy injection until the date and time of the clinic visit at which complete resolution of all local signs and symptoms was documented, i.e., the lesion had aborted or complete healing had occurred (censored at Day 10), thereby including patients both with classical episodes and with aborted episodes. (The time of the final Day 10 visit was used for primary endpoint analysis in subjects censored at Day 10.) For patients experiencing classical episodes, complete healing was defined as “the absence of crust, with no evidence of active lesion, whether or not there were any residual post-lesion skin changes which might include erythema, flaking, or slight asymmetry.”

Secondary endpoints included the time from treatment initiation to 1) complete healing of classical episodes (episodes which progressed to the vesicular or later stages; censored at Day 10); 2) episode abortion, 3) complete cessation of pain; and 4) the proportion of aborted episodes, defined as episodes which did not progress beyond the papule stage. Aborted episodes were considered healed at the time of the clinic visit where cessation of HSL-related signs or symptoms was reported.

Safety and tolerance of topical n-docosanol 10 wt. % cream were determined by adverse experience reports and assessment of clinical laboratory variables.

The sample size for the combined study was based on data from prior clinical studies. The combined study was planned to have 700 evaluable patients (350 per group), that would allow the detection of a 13-hour mean difference between treated and placebo groups with 82% power. The two sub-studies were also analyzed separately.

Statistical methodologies were outlined in the protocol. The intent-to-treat (ITT) population included all patients who received medication and had at least one treatment evaluation. The efficacy evaluable population was protocol adherent and applied at least 80% of scheduled doses. Protocol deviations were evaluated prior to study unblinding. The safety evaluable population included all those who used at least one application of study medication.

Demographic and medical history data were tabulated by treatment group and descriptive statistics were used for continuous variables. Frequencies and proportions were used for categorical variables. Baseline variables such as signs and symptoms, location of prodrome, current experience, and lesion stage were compared for homogeneity between randomized treatment groups using either analysis of variance or Cochran-Mantel-Haenszel tests. (See Agresti A. An introduction to categorical data analysis. New York: Wiley 1996; pp. 60-4). Descriptive statistics for baseline vital signs were calculated.

For the primary efficacy analyses all patients who had at least one post-baseline efficacy assessment were included. Time-to-event distributions were estimated by Kaplan-Meier product-limit estimates. (Kaplan E L, Meier P. J. Am. Stat. Assoc. 53:457-81, 1958). Time-to-event distributions were compared between treatments using the Gehan generalization of the Wilcoxon test, stratified by site. (Gehan E A Biometrika 52:203-23, 1965). In consultation with FDA, the Generalized Wilcoxon test was chosen because it has good power when the effects of treatment are expected early in the treatment period. Confidence intervals (hours of difference) were obtained by numerical inversion of the stratified Wilcoxon test. Lesion assessments from participants whose lesions were unhealed at 10 days were censored at that point. The percentage of aborted episodes is presented by latest stage at baseline visit.

Possible adjustment for important baseline covariates was identified in the protocol. Because the Generalized Wilcoxon test does not readily allow for adjustments for covariates, proportional hazards regression (Cox regression) was used as a means of gauging whether covariate adjustment would have an effect on the p-value for treatment. All p-values reported represent the unadjusted analysis.

In the combined study, seven hundred forty-three subjects were randomized at twenty one U.S. sites. Three hundred seventy-three individuals were randomized to receive n-docosanol while three hundred seventy were randomized to receive placebo. Three n-docosanol-treated and three placebo-treated patients (0.8% of the study population) did not return to the clinic after the initial visit. These six patients were included in the safety analysis, however, per protocol design, they were excluded from the intent-to-treat efficacy population. The efficacy evaluable population was nearly identical to the intent-to-treat population—97.4% of randomized patients were efficacy evaluable. As such, only the data from the ITT population are discussed.

In sub-study #06 eight sites randomized three hundred seventy patients, one hundred eighty-five to n-docosanol and one hundred eighty-five to placebo. In sub-study #07 thirteen sites randomized three hundred seventy three patients, 188 to n-docosanol and to placebo.

Patient demographic and baseline characteristics for the ITT population of the combined study are presented in Table 7. The demographics of the individual studies were similar and are not shown. There were no significant differences between treatments in race, age, or frequency of HSL recurrences. The mean age of study patients was 37 years with a range of 18 to 80 years. Minor gender differences were identified. The majority of study participants were female and Caucasian, however, males comprised a smaller proportion of the n-docosanol recipients compared to placebo recipients (25% versus 33%, respectively; p=0.01). At enrollment all recurrent episodes were less than 12 hours in duration. Between 75 and 80% of patients presented for treatment with erythema, with the remainder presenting with prodrome only. This distribution was similar in both treatment groups (See also Table 10). Pain reported at baseline also did not differ between treatment groups.

Past experience with HSL as obtained by patient report at the baseline visit is also summarized in Table 7 for the combined study. Between treatment groups there were no statistically significant differences in the time since first onset of HSL or the time since the last HSL episode, the number of episodes in the previous year, the proportion of participants who usually experience localized prodrome or the duration of the most recent HSL episode. n-Docosanol recipients, however, reported a longer history mean episode duration compared with placebo recipients (9.5 versus 8.4 days, respectively; p=0.02). This statistical difference was also observed in study #06 (10.1 days and 8.4 days, respectively; p=0.01). The mean duration of the most recent previous episode (10.0 versus 8.4 days; p=0.02, n-docosanol versus placebo) was also statistically different in study #06. No treatment group differences in HSL history were observed in study #07. Where statistical differences were observed in study demographics between treatment groups, Cox regression analysis was utilized to assess the covariate affect.

This was an experienced HSL population. Participants reported a median of five episodes in the past 12 months with a mean HSL history greater than 20 years. More than 99% of participants reported that they normally experience prodromal symptoms prior to their HSL episodes.

The mean number of applications for the n-docosanol group was 24.1 and the mean number for the placebo group was 25.7. Treatment compliance was assessed by comparing the number of applications actually made to the number that should have been made and averaged 99.2% in the n-docosanol group and 99.6% in the placebo groups. There were no statistically significant differences between treatment groups with respect to the number of applications or compliance.

TABLE 7
PATIENT CHARACTERISTICS AND
HISTORICAL INFORMATION FOR ITT
POPULATION COMBINED STUDY 06/07

| | Docosanol | Placebo | |
|---|---|---|---|
| Parameter | (N = 370) | (N = 367) | p-values^{a} |
| Gender | | | 0.007 |
| Male | 91 (24.6%) | 122 (33.2%) | |
| Female | 279 (75.4%) | 245 (66.8%) | |
| Race | | | NS^{b} |
| Caucasian | 348 (94.1%) | 345 (94.0%) | |
| Black | 10 (2.7%) | 13 (3.5%) | |
| Asian | 2 (0.5%) | 1 (0.3%) | |
| Hispanic | 8 (2.2%) | 4 (1.1%) | |
| Other | 2 (0.5%) | 4 (1.1%) | |
| Age (years) | | | NS |
| N | 370 | 367 | |
| Mean (SD) | 37.2 (12.8) | 37.4 (13.4) | |
| Range | 18-77 | 18-80 | |
| Stage of Lesion at Baseline | | | NS |
| Prodrome | 71 (19.2%) | 80 (21.8%) | |
| Erythema | 299 (80.8%) | 287 (78.2%) | |
| Average Episode-Duration from | | | 0.016 |
| Patient History (days) | | | |
| N | 370 | 367 | |
| Mean (SD) | 9.5 (4.2) | 8.4 (3.7) | |
| Range | 1-42 | 1-30 | |
| Previous Duration of Most | | | NS |
| Recent Episode (days) | | | |
| N | 370 | 367 | |
| Mean (SD) | 9.1 (5.0) | 8.2 (4.2) | |
| Range | 1-60 | 1-30 | |
| Time since Last Onset of | | | NS |
| Oral-Facial Herpes | | | |
| Simplex (months) | | | |
| N | 369 | 366 | |
| Mean (SD) | 3.0 (2.2) | 3.0 (2.2) | |
| Range | 0-11 | 0-12 | |
| Time since First Onset of | | | NS |
| Oral-Facial | | | |
| Simplex (years) | | | |
| N | 370 | 366 | |
| Mean (SD) | 22.4 (13.8) | 21.4 (13.2) | |
| Range | 0-68 | 0-64 | |
| Number of Episodes in | | | NS |
| Past 12 Months | | | |
| N | 370 | 367 | |
| Mean (SD) | 5.2 (3.7) | 5.1 (3.1) | |
| Range | 2-40 | 2-20 | |
| Does Patient Experience | | | NS |
| Localized Prodrome? | | | |
| No | 3 (0.8%) | 1 (0.3%) | |
| Yes | 367 (99.2%) | 366 (99.7%) | |

^{a}P-value for categorical parameters from Cochran-Mantel-Haenszel test adjusted for site. P-value for continuous parameters from analysis of variance model with effects for treatment, site, and site-by-treatment interaction.
^{b}Not significant

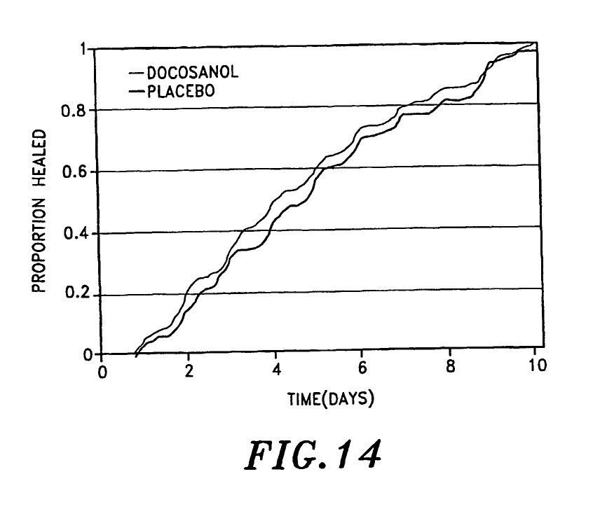

Efficacy data are summarized for both the combined study and each sub-study in Table 8. Only the combined study results are discussed in the text. The vast majority of participants healed during the 10-day treatment period (91% of n-docosanol recipients and 90% of placebo recipients). Kaplan-Meier curves for times to healing are displayed in FIG. 14. The median time-to-complete-healing for all lesions was 4.08 days for n-docosanol recipients versus 4.80 days for placebo recipients, a difference of 15% (p=0.008; 95% Cl2, 22 h). The distribution of healing times also favored n-docosanol treatment at the 25th and 75th percentiles.

Covariate adjustment utilizing proportional hazards regression for differences in the number of males had no affect on the p-value for time-to-healing; however, for historical episode duration the p-value decreased (i.e., became more significant).

TABLE 8
EFFICACY ENDPOINTS FOR ITT POPULATION

| | Combined Study 06/07 | | | Study 06 | | | Study 07 | | |
|---|---|---|---|---|---|---|---|---|---|
| | | Docosanol | | | Docosanol | | | Docosanol | |
| | Median | Median | p-value^{c} | Median | Median | p-value^{c} | Median | Median | p-value^{c} |
| Parameter | Difference^{a} | Time^{b} | (95%Cl) | Difference^{a} | Time^{b} | (95% Cl) | Difference^{a} | Time^{b} | (95% Cl) |
| Time-to-healing | 17.5 hr | 97.8 hr | 0.008 | 18.9 hr | 94.9 hr | 0.023 | 15.9 hr | 102.3 hr | 0.153 |
| (All episodes) | | | (2, 22) | | | (1.5, 25.75) | | | (−2.25, 23.75) |
| Time-to-healing | 1.6 hr^{d} | 142.1 hr | 0.023 | 0.5 hr | 137.8 hr | NS^{e} | 22 hr | 143.0 hr | 0.021 |
| (Classical episodes) | | | (1, 24.5) | | | (−4.25, 24.25) | | | (1, 37.5) |
| Hours to cessation of | 13.4 hr | 52.3 | 0.002 | 12.8 hr | 52.3 hr | 0.02 | 12.9 hr | 52.9 hr | 0.03 |
| pain and all | | | | | | | | | |
| symptoms (burning, | | | (3, 16.5) | | | (1.25, 18.25) | | | (0.5, 19.75) |
| itching, tingling) | | | | | | | | | |

Medians are based on Kaplan-Meier estimates.
^{a}Difference between n-docosanol and placebo in median time-to-event
^{b}Median Time to event for the n-docosanol treated group
^{c}P-value from Gehan generalized Wilcoxon test stratified by site
^{d}At the 25^{th} and 75^{th} percentiles, the difference was approximately 19 h
^{e}Not significant

Approximately 60 to 65% of subjects developed classical episodes. The difference in time-to-healing (Table 8) was statistically shorter in the n-docosanol-versus the placebo-treated groups (p=0.02; 95% Cl1, 24.5 h). For this endpoint, larger differences were observed at the 25th and 75th percentiles (˜19 h) than at the median (1 h).

Values for the time-to-cessation of individual lesion stages for classical episodes are displayed in Table 9. The median for time-to-cessation of vesicles was approximately 2.1 days and the median for time-to-cessation of hard crusts was approximately 5.8 days. Neither was statistically different between treatment groups. However, the median time-to-cessation of the ulcer/soft crust stage was shorter in the n-docosanol group (3.61 versus 3.94 days; p<0.001; 95% Cl 8, 25 h).

TABLE 9
TIME-TO-CESSATION OF DISCRETE LESION STAGES
FROM CLASSICAL EPISODES

| | Combined Study 06/07 | | | Study 06 | | | Study 07 | | |
|---|---|---|---|---|---|---|---|---|---|
| | | | p-value^{a} | | | p-value^{a} | | | p-value^{a} |
| Parameter | Docosanol | Placebo | (95% Cl) | Docosanol | Placebo | (95% Cl) | Docosanol | Placebo | (95% Cl) |
| Median hours to cessation | 50.5 | 50.7 | NS^{b} | 49.4 | 49.9 | NS | 50.9 | 53.5 | NS |
| of the vesicular stage (from | | | (−1.75, 9) | | | (−3.5, 9.75) | | | (−3, 15.75) |
| initiation of treatment) | | | | | | | | | |
| Median hours to cessation | 86.7 | 94.5 | <0.001 | 76.5 | 89.0 | 0.014 | 92.7 | 100.8 | 0.007 |
| of the ulcer/soft crust stage | | | (8, 25) | | | (2.25, 24.25) | | | (4.75, 40.25) |
| (from initiation of treatment) | | | | | | | | | |
| Median hours to cessation | 142.8 | 142.3 | NS | 138.8 | 138.3 | NS | 146.0 | 145.3 | NS |
| of the hard crust stage (from | | | (−2, 21) | | | (−5.75, 23.5) | | | (−5.25, 26.75) |
| initiation of treatment) | | | | | | | | | |

^{a}Medians are based on Kaplan-Moier estimates.
^{a}P-value from Gehan generalized Wilcoxon test stratified by site
^{b}Not significant

A total of seven hundred five (96%) of the seven hundred thirty seven patients in the ITT group, equally distributed between placebo and n-docosanol-treated populations, experienced lesion pain and/or burning, itching, or tingling during the study. Median times to complete cessation of pain and/or burning, itching, or tingling for all participants (Table 9) was 2.18 days for n-docosanol recipients versus 2.74 days for placebo recipients (approximately 20% reduction; p=0.002; Cl 3, 16.5 h).

Results for patients with aborted episodes by stage at baseline are summarized in Table 10. For all subjects, a trend (not statistically different) toward more aborted episodes was identified with 39.7% n-docosanol recipients experiencing aborted episodes versus 34.1% placebo recipients (p=0.109; Cl for odds ratio 0.95, 1.73). For sub-study #06, in subjects who began treatment with erythema, 34.3% of n-docosanol recipients versus 23.3% of placebo recipients (p=0.048; Cl 1.00, 2.75) experienced aborted episodes. The times to episode abortion were rapid and not different between treatment groups.

TABLE 10
PERCENT OF PATIENTS WITH ABORTED EPISODES
BY STAGE AT BASELINE

| | Combined Study 06/07 | | | Study 06 | | | Study 07 | | |
|---|---|---|---|---|---|---|---|---|---|
| | | | p-value^{a} | | | p-value^{a} | | | p-value^{b} |
| Stage at Baseline | Docosanol | Placebo | (95% Cl) | Docosanol | Placebo | (95% Cl) | Docosanol | Placebo | (95% Cl) |
| All patients (prodrome or | 39.7% | 34.1% | 0.109 | 38.8% | 30.1% | 0.078 | 40.6% | 38.0% | NS |
| erythema at baseline) | N = 370^{b} | N = 367 | (0.95, 1.73) | N = 183 | N = 183 | (0.96, 2.24) | N = 187 | N = 184 | (0.73, 1.73) |
| Prodrome at baseline | 63.4% | 52.5% | NS^{c} | 55.0% | 48.0% | NS | 74.2% | 60.0% | NS |
| | N = 71 | N = 80 | (0.65, 2.76) | N = 40 | N = 50 | (0.55, 3.02) | N = 31 | N = 30 | (0.37, 5.71) |
| Erythema at baseline | 34.1% | 28.9% | NS | 34.3% | 23.3% | 0.048 | 34.0% | 33.8% | NS |
| | N = 299 | N = 287 | (0.88, 1.78) | N = 143 | N = 133 | (1.00, 2.75) | N = 156 | N = 154 | (0.59, 1.59) |

^{a}P-value from Cochran-Mantel-Haenszel test adjusted for center. Confidence intervals are given for the odds ratio, adjusted for center. Odds ratios larger than 1.00 indicate that n-docosassl patients are more likely than placebo patients to have an aborted episode.
^{b}N = total number of patients evaluated
^{c}Not significant

Adverse experiences were quantitatively and qualitatively similar between n-docosanol-treated and placebo-treated patents. At least one adverse experience was reported by 19.6% (73/373) of n-docosanol recipients and 18.9% (70/370) of placebo recipients for the combined study population. Headache, which was reported by 5.9% of patients in each treatment group, was the most common adverse experience. With the exception of application site reaction (2.1% of the n-docosanol group and 1.9% of the placebo group) and herpes simplex outside of the treatment area (2.4% of the n-docosanol group and 1.4% of the placebo group), all adverse experiences were reported by less than 2% of the patients in either treatment group. Two patients, (one patient in each group) were withdrawn from the study due to adverse experiences of rash and herpes simplex outside the treatment area, respectively. There were no statistically significant differences between treatment groups with respect to change from baseline in either hematology or clinical chemistry parameters.

This trial with n-docosanol 10% cream demonstrates clinical efficacy of early clinic-initiated therapy of recurrent HSL. The combined study analysis showed statistically significant reductions in time-to-complete-healing, time-to-complete-healing of classical episodes, cessation of the most active infectious lesion stage (ulcer/soft crust) and cessation of all HSV symptoms. Median time-to-healing was the primary efficacy parameter and was reduced by 0.72 day compared with placebo. The times-to-healing of classical lesions and the times-to-cessation of ulcer/soft crust were also significantly reduced. The ulcer/soft crust stage represents the peak period of viral replication and inflammation, which may explain its sensitivity of response.

The statistical differences identified in the individual sub-studies (#06 and #07) were slightly less robust than in the combined study, reflective of fewer participants. The sub-studies were similar in treatment effects to the combined study and to each other. Consistency of the results across the sub-studies was analyzed utilizing various methods of analysis and measures of effect including Proportional Odds Regression, Proportional Hazards Regression, and Log-Logistic Regression models (results not shown) in addition to the Generalized Wilcoxon reported here. The estimated treatment effects are very similar regardless of the measure of effect used. Furthermore, confidence intervals for treatment effects computed are almost completely overlapping.

The combined analysis approach for the sub-studies was planned by the protocol. The two studies combined represented a cohort size approximately half that reported for each of two topical penciclovir cream studies in HSL; neverheless, the studies demonstrated clinical and statistical significance for n-docosanol against both the [symbol] ng and symptom components of HSL. (For a discussion of topical penciclovir cream studies in HSL, see Spruance S K, Rea T L, Thoming C, Tucker R, Saltzman R, Boon R JAMA 277:1374-9, 1997; and Rabom G W 36th Interscience Conference on Antimicrobial Agents and Chemotherapy, New Orleans, 1996). Using this early, clinic-initiated model with twice daily observations, a cohort of 700 to 800 patients appears sufficient to demonstrate these key efficacy components of HSL treatment. In contrast, demonstration of lesion prevention may require a larger patient population. Despite the interesting trends in favor of n-docosanol treatment, this study was not sufficiently powered to demonstrate lesion prevention at the rates observed and, unfortunately, to date, lesion prevention has never been unequivocally demonstrated. Nevertheless, clinic-initiated treatment prior to lesion onset clearly offers the potential for demonstration of this treatment benefit (given the proper cohort size) where it exists. No other reported study design truly provides the opportunity to demonstrate such effects since a high proportion of patents who self-initiate therapy in the prodromal phase may actually have early, established lesions prior to commencement of therapy. (Spruance S L, Overal J C, Kern E, Krueger G G, Pliam V, Miller W New Engl. J. Med. 297:69-75, 1997; and Spruance S L Semin. in Dermatol. 11:200-6, 1992).

Penciclovir cream 1% is currently available by prescription for the topical treatment of recurrent herpes simplex labialis. Based on information from the product insert for penciclovir cream, in the US multicenter study more than twice the size of the current study, Spruance et al. demonstrated that penciclovir-treated patients experienced a significantly shorter mean time to healing with a 0.5 day difference (4.5 versus 5.0 days; p<0.001). (See Spruance S K, Rea T L, Thoming C, Tucker R, Saltzman R, Boon R JAMA 277:1374-9, 1997). Lesion pain was reduced, as demonstrated by an approximately half-day reduction in the mean duration of lesion pain (3.9 versus 4.4 days; p<0.001). Spruance et al. reported viral shedding was reduced by penciclovir as demonstrated by changes over the shedding period followed (vesicle and ulcer/soft crust), although no differences in median times to loss of viral shedding were observed (3.0 versus 3.0 days). The difficulty in demonstrating an antiviral effect with penciclovir cream, given the large numbers of subjects tested, suggests that viral cultures must be aggressively obtained in order to make this the sensitive efficacy marker it has been in studies of herpes genitalis. (See Diaz-Mitoma F, Ruben M, Sacks S L, MacPhersom P, Caissie G. J. Clin. Microbiol. 34:657-63, 1996; Sacks S L, Aoki F Y, Diaz-Mitoma F, Sellors J, Shafran S D JAMA 276:44-9, 1996; and Sacks S L, Tyrrell D L, Lawee D, Schlech W, Gill M J, Aoki F Y, et al. J. Infect. Dis. 164:665-72, 1991). Aggressive viral culturing has often not been pursued in HSL because of a possible effect on delaying healing, which may, in turn, contribute to the lack of sensitivity of this parameter in HSL studies. Accordingly, viral cultures were not performed in the current studies.

As observed in the penciclovir studies, the placebo treated time of about 5 days in these studies is shorter than the reported natural history of HSL lesion healing of 7 to 10 days. (Spruance S L, Overal J C, Kern E, Krueger G G, Pliam V, Miller W New Engl. J. Med. 297:69-75, 1997; Spruance S L. Semin. in Dermatol. 11:200-6, 1992; and Shafran S D, Sacks S L, Aoki F Y, Tyrrell D L, Schlech W F 3^{rd}, Mendelson J, Rosenthal D, et al. J. Infect. Dis. 176:78-83, 1997). This raises suspicions of a placebo effect, which has been well recognized in HSL. (Spruance S L In: Clinical management of herpes viruses, Sacks S L, Straus S E, Whitley R J, Griffiths P D, editors, Amsterdam: IOS Press, p. 3-42, 1995; and Guinan M E, MacCalman J, Kern E R, Overall J C Jr., Spruance S L JAMA 243:1059-61, 1980). Placebo effects often occur with dermatological products, resulting not only from the psychological effects typically associated with placebo treatment, but also due to simply covering the lesion which itself alters the physiology of untreated skin. (Placebo effects are discussed in Chaput de Saintonge D M, Herxheimer A Lancet 344:995-8, 1994).

Although the effect of n-docosanol 10% cream in HSL may appear modest, the self-limiting nature of the disease makes decreased duration of almost a day (18 h) significant to patients. Additionally, the apparent magnitude of the clinical effect may be lessened by what appears to be a substantial placebo effect in the treatment of HSL, as discussed above. Reduced healing time is accompanied by relief of pain and/or burning, itching or tingling, also important to patients. The time of the most severe stage (ulcer/soft crust) of the lesion is significantly reduced, a medically important effect that has not been reported previously. Its approval as an over-the-counter (OTC) product allows it to be applied early in the course of an episode where it is most likely to be effective.

Docosanol appears to inhibit viral entry into host cells by inhibiting the normal process of viral fusion with the cell's plasma membrane thus blocking entry and subsequently limiting viral replication. n-Docosanol and its metabolites do not interact directly with viral proteins or nucleic acids. Accordingly, the emergence of drug-resistant HSV is unlikely. Because of the different mode of action from antiviral nucleosides, resistance to n-docosanol would not diminish the effectiveness of other topical or systemic antivirals, even if it were shown to exist. In addition, the unique mechanism of action suggests that combination therapy with antiviral nucleosides is worthy of consideration.

In summary, n-docosanol 10% cream was shown to be effective in this clinic-initiated, placebo controlled, clinical trial in early HSL. This treatment reduced episode duration overall, duration of those episodes that developed into classical lesions, and the duration of all lesion symptoms. Based on these studies, treatment with n-docosanol 10 wt. % cream should be initiated as early as possible in the course of HSL.

A study was conducted to examine the in vivo efficacy of 10% docosanol in two formulations using guinea pig models. Clinical trials have demonstrated efficacy of docosanol 10% cream (“doc”) in the treatment of herpes simplex labialis (HSL) leading to FDA approval in July 2000. Docosanol exhibits antiviral activity in vitro, but inconsistent results have been reported in animal HSV models.

Hairless and Hartley (shaved and treated with Nair) guinea pigs were inoculated at 6-8 sites on the back with HSV-1 (3×10 PFU Macintyre or KOS) or HSV-2 (2×10 PFU MC) with an electric tattoo marker or by scratching with a 20 gauge needle. Treatments with doc or vehicles (“veh”) began 12 hours later and continued 3-4 times daily. At the peak of viral titer, animals were scanned, lesions removed and virus titer determined by assessing cytopathic effect of homogenates in Vero cells.

In hairless guinea pigs antiviral activity of doc against HSV-1 and HSV-2 was shown by reduced vesicle number following inoculation with a tattoo marker (e.g., for HSV-2 mean veh=21.4±2.4 vs mean doc=12.7±1.9; p<0.01) and reduced viral titers (e.g., for HSV-1 mean log veh=4.0 vs mean log doc=3.6; p<0.001 and for HSV-2 mean log veh=5.84 vs mean log doc=4.84; p=0.002) for both inoculation methods. There was no reduction in lesion score or size following inoculation by scarification. In Hartley guinea pigs HSV-1 and HSV-2 vesicle numbers were inhibited (e.g., HSV-2 mean log veh=26.7±8 vs mean log doc=7.7±4.8; p<0.002). Inhibition of viral titer was less than in hairless animals but was statistically significant in some experiments (HSV-1 mean log veh=6.38 vs mean log doc=5.79; p<0.001 and for HSV-2 mean log veh=5.47 vs mean log doc=5.07; p=0.06). Formulation differences were noted.

Doc reduces HSV-1 and HSV-2 viral titers in hairless guinea pigs and vesicle numbers in both hairless and Hartley guinea pigs. The difference in efficacy in the two models may explain previous varying results with doc in animal models despite its demonstrated clinical efficacy. The hairless model, including inoculation with a tattoo gun, may be a better model of clinical HSV infections.

As discussed above, n-docosanol is a saturated 22-carbon primary alcohol that inhibits HSV replication in tissue culture. See, e.g., Katz et al., “Antiviral activity of 1-docosanol, an inhibitor of lipid-enveloped viruses including herpes simplex,” Proc. Natl. Acad. Sci. (1991) 88:10825-9; and Pope et al., “The anti-herpes simplex virus activity of n-docosanol includes inhibition of the viral entry process,” Antivir. Res. 40:85-94 (1998). It has also been demonstrated to shorten the duration of disease in experimental animals. See Marcelletti et al., “Docosanol” 17:879-82 (1992). Clinical trials have demonstrated efficacy of docosanol 10% cream (doc) in the treatment of herpes simplex labialis. See Habbema et al., “n-Docosanol 10% cream in the treatment of recurrent herpes labialis,” Acta Derm. Venereol. 76:479-81 (1996). In a large double-blind study, the median time-to-healing in the three hundred seventy docosanol-treated patents was 4.1 days, 18 hours shorter than observed in the 367 placebo-treated patients (p=0.008). See Sacks, et al., “Clinical efficacy of topical docosanol 10% cream for herpes simplex labialis: A multicenter, randomized, placebo-controlled trial,” J. Amer. Acad. Derm. 45:222-30 (2001). The docosanol group also exhibited reduced times from treatment initiation to: 1) cessation of pain and all other symptoms (itching, burning and/or tingling, p=0.002); 2) complete healing of classical lesions (p=0.023); and 3) cessation of the ulcer/soft crust stage of classical lesions (p<0.001). FDA approved docosanol 10% cream as an over-the counter treatment for cold sores in July 2000. Docosanol inhibits in vitro a broad spectrum of lipid-enveloped viruses including HSV-1 and HSV-2, cytomegalovirus, varicella zoster virus and human herpes virus. Data suggest that after cellular incorporation and metabolic conversion, docosanol inhibit viral entry by inhibiting viral fusion with the host cell, blocking nuclear localization and subsequent replication of virus. See Pope et al., “Anti-herpes simplex virus activity of n-docosanol correlates with intracellular metabolic conversion of the drug,” J. Lipid Res. 77:2167-78 (1996). This mechanism of action is different from that of other available treatment options for herpes infections, where antiviral activity results from inhibition of DNA synthesis. See Elion, “Acyclovir: discovery, mechanism of action, and selectivity,” J. Med. Virol. 1:2-6 (1992).

Docosanol and acyclovir were prepared in two types of formulations: a cream formulation and a polyethylene glyco-(PEG) based ointment. The compositions of both formulations are listed in Table 11a (Composition of Docosanol and Acyclovir Creams) and Table 11b (Composition of Docosanol and Acyclovir in PEG).

TABLE 11a

| | Formulation (% w/w) | | |
|---|---|---|---|
| | | Docosanol | Acyclovir |
| Ingredient | Vehicle | 10% Cream | 5% Cream |
| Docosanol | 0 | 10 | 0 |
| Acyclovir | 0 | 0 | 5 |
| Cream excipients | 100 | 90 | 95 |

TABLE 11b

Formulation (% w/w)

| | PEG | Docosanol | Acyclovir |
|---|---|---|---|
| Ingredient | Vehicle | 10% in PEG | 5% in PEG |
| Docosanol | 0 | 10 | 0 |
| Acyclovir | 0 | 0 | 5 |
| PEG 3350 | 30 | 15 | 20 |
| PEG 400 | 70 | 75 | 75 |

Hairless and Hartley guinea pigs (Crl:(HA)BR) were obtained from Charles River laboratories. They were quarantined 7 days before use and fed diet and water ad libitum. The animals were individually caged and housed under strict pathogen-free conditions. Two strains of HSV-1 (Kos strain and MacIntyre strain) and the MS stain of HSV-2 were used. The virus was a cell culture preparation that had been pre-titered in guinea pigs prior to use in these experiments.

Prior to inoculation the haired guinea pigs were shaved with an electric razor, dampened with warm water, then Nair depilatory cream was applied for 3-4 minutes to remove the remaining hair. The backs of both hairless and haired animals were then washed with warm water and thoroughly dried. In Inoculation Method 1, the backs of guinea pigs were marked into a grid of 8 squares and within each area a 10 mm diameter lesion (wound) was induced by applying virus to the skin and scarifying the area with 10 light vertical and horizontal scratches using a 20 gauge inoculation needle. In Inoculation Method 2, a grid of six squares was drawn with a marking pen. Each square was inoculated with 50-75 l volume of virus with an electric tattoo gun (Spaulding and Rogers, Inc., Voorheesville, N.Y.). The instrument was triggered 80 times at each inoculation site with the dial set at 17.

With Inoculation Method 1 (scarification) the length and width of each lesion (wound) is measured, and the lesion is assigned a score daily that ranges from 0 (normal) to 4 (maximal). These measurements were made up until the time of sacrifice on Day 4 (Hartley guinea pig). Vesicles may form within the lesion, but these were not counted. With the tattoo inoculation method (Method 2), discrete vesicles are formed, and there is no wound between the vesicles. With this inoculation method vesicles were counted and recorded, and the total involved area was not determined.

Animals were sacrificed at the peak of viral titer. Skin containing each lesion was removed from the sacrificed animals and homogenized in an approximately 10% w/v suspension in MEM containing 2% FBS, 0.18% NaHCO₃, and 50 g/ml gentamicin. Serial dilutions were assayed in triplicate wells in 96-wells plates containing a 24-h monolayer of Vero cells. The plates were sealed, incubated for 7 days at 37° C., and then examined under a microscope for discernible viral cytopathic effect.

For the experiment described in Table 12, Student's t-test was used to compare mean lesion size and mean lesion virus titers. Ranked sum analysis was used to evaluate lesion score. For all other experiments one-way Analyses of Variance (ANOVA) for treatment as the factor were performed separately for each study. If the ANOVA was significant, then the least squares (LS) means for lesion viral titers and vesicle numbers were calculated and unadjusted multiple comparisons were performed testing for differences in these means for all pairs of treatments.

TABLE 12
Effect of Topical Therapies on HSV-1 Induced Lesions in Hairless Guinea Pigs

| | | | | | Mean Virus Titer |
|---|---|---|---|---|---|
| | Mean Lesion Score ± SD | | Mean Lesion Size ± SD | | Day 4 |
| Treatment | Day 2 | Day 3 | Day 2 | Day 3 | (log₁₀/g ± SD) |
| 10% Docosanol | 0.7 ± 0.3 | 0.8 ± 0.3 | 32.9 ± 14.5 | 40.8 ± 18.4 | 3.7 ± 1.2** |
| (PEG) | | | | | |
| 10% Docosanol | 0.6 ± 0.3 | 0.8 ± 0.3 | 34.2 ± 16.1 | 43.3 ± 18.3 | 3.6 ± 1.3** |
| (Cream) | | | | | |
| 5% Acyclovir | 0.6 ± 0.3 | 0.8 ± 0.3 | 25.8 ± 13.6 | 35.0 ± 15.7 | 4.0 ± 1.1** |
| (PEG) | | | | | |
| 5% Acyclovir | 0.4 ± 0.2 | 0.4 ± 0.2▪▪** | 13.2 ± 10.3▪*** | 13.6 ± 9.0**▪▪▪ | 3.1 ± 0.8**▪▪▪ |
| (Cream) | | | | | |
| Cream Vehicle | 0.5 ± 0.2 | 0.5 ± 0.3 | 24.0 ± 11.5 | 29.3 ± 15.7 | 4.9 ± 0.8 |
| PEG Vehicle | 0.7 ± 0.2 | 0.8 ± 0.3 | 33.7 ± 14.8 | 38.0 ± 13.7 | 4.7 ± 0.8 |
| Untreated | 0.6 ± 0.2 | 0.8 ± 0.3 | 28.2 ± 9.5 | 38.6 ± 14.1 | 5.0 ± 0.7 |

▪tid x 4 beginning 12 h post virus exposure
*P < 0.05 **P < 0.01 ***P < 0.001 compared to appropriate placebo
*P < 0.05 **P < 0.01 ***P < 0.001 compared to untreated controls

The formulations prepared are listed in Tables 11a and11b above. All samples were subjected to analytical testing prior to experimental use. The cream formulation with docosanol is a white, odorless, non-staining and non-water soluble cream. In the absence of docosanol, the cream vehicle and 5% acyclovir in cream vehicle have watery, lotion-like consistencies. The PEG vehicle is a clear, water-soluble ointment that becomes white in formulations containing docosanol and acyclovir.

The formulations listed in Tables 11a and 11b were evaluated for efficacy in the treatment of cutaneous lesions induced by HSV-1 in hairless models using Inoculation Method 1 (scarification). Topical treatment began 12 hours later and continued every 8 hours for a total 10 treatments. Lesion size and severity were assessed on Day 2 and Day 3 of infection. On Day 4 each lesion was excised and assayed for viral content.

The results are summarized in Table 12. Lesion size and score were not inhibited by docosanol in cream or ointment or by acyclovir ointment. It has been reported that greater inhibition of lesion size and severity is generally observed if treatment is continued past Day 4, and since guinea pigs in this study were sacrificed on Day 4 for determination of viral content, it was not unexpected that effects on lesion size and severity were not observed. The virus titer reduction data indicated that docosanol treatment in both vehicles reduced the mean virus titer/gram by approximately 1 log₁₀ when compared to the untreated control mean. This difference was statistically significant (p<0.01). Docosanol in PEG reduced the viral titer by 1.0 log₁₀ and acyclovir in PEG reduced the viral titer by 0.7 log₁₀. The differences between acyclovir and docosanol were not statistically significant.

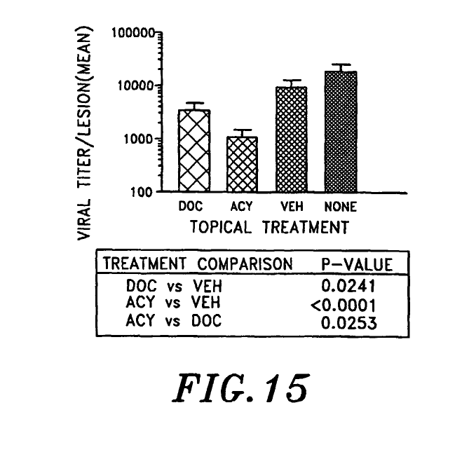

Based on the results in Table 12, and because the PEG vehicle is similar in consistency to that of docosanol in PEG, PEG formulations were selected for further study. The results of tests in hairless guinea pig with inoculation of virus with Method 2 are illustrated in FIG. 15. Viral titer levels were statistically significantly reduced compared to vehicle treated sites (mean log₁₀=4.0) by docosanol (mean log₁₀=3.5) and acyclovir (mean log₁₀=3.0).

Hairless guinea pigs were inoculated with the MacIntyre strain HSV-1 (60 μl 5×10 PFU/ml) with tattoo inoculation on each of 8 sites on the dorsal surface. Treatments were started twelve hours post inoculation and were repeated 3 times per day for 3 days. Lesion skin was collected on Day 4, at 12 hours after the last treatment and assayed for viral content. The treatment groups were doc=10% docosanol in PEG (10 sites), acy=5% acyclovir in PEG (10 sites), veh=PEG vehicle (10 sites), and none=no treatment (8 sites).

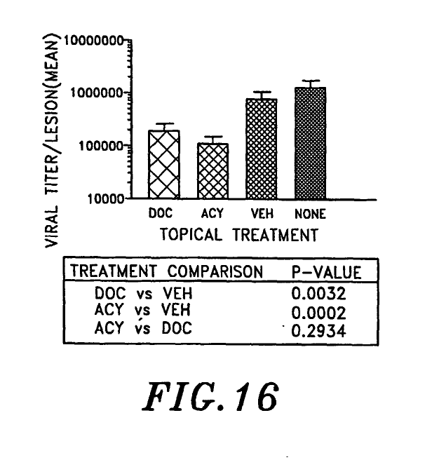

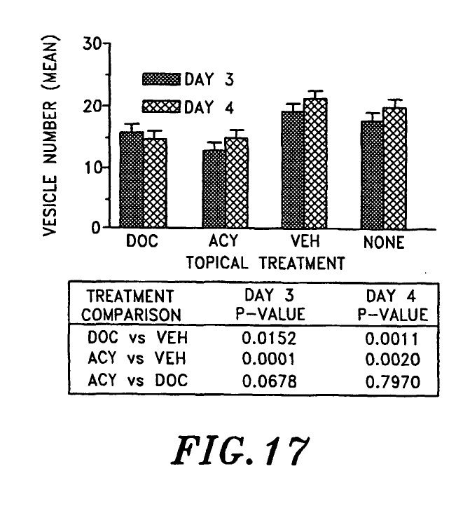

Similar observations were made following inoculation of hairless guinea pigs with HSV-2 (MS strain). HSV-2 viral titer levels were statistically significantly reduced compared to PEG vehicle treated sites (mean log₁₀=5.8) by a topical treatment with docosanol in PEG (mean log₁₀=5.2) and acyclovir in PEG (mean log₁₀=5.0). Viral titers were reduced 75% (docosanol) and 78% (acyclovir) following topical treatment (FIG. 16). The numbers of vesicle/site on Day 3 and Day 4 after infection were also assessed and are shown in FIG. 17.

Hairless guinea pigs were inoculated with the MS strain of HSV-2 (60 μl 1×10 PFU/ml) utilizing a tattoo gun as described in Materials and Methods on each of 6 sites on the dorsal surface. Treatments started twelve hours post inoculation and were repeated every 8 hours for 3 days. Vesicle numbers were counted on Days 3 and 4. Lesion skin was collected on Day 4, 12 hours after the last treatment and assayed for viral content. All treatments were applied to 9 sites except that only 3 sites received no treatment.

The disease duration in the hairless guinea pig models is 4-5 days after inoculation with virus. The duration of disease with the haired guinea pig is 8-9 days. The longer disease duration provides two advantages: 1) it better represents the disease course in humans of 8 to 10 days for herpes labialis and 7 to 10 days for herpes genitalis in both men and women and 2) it provides a larger window to observe a therapeutic effect. See Spruance “The natural history of recurrent oral-facial herpes simplex virus infection,” Semin. Dermatol. 11:200-6 (1992); Whitley et al. “Herpes simplex virus infections,” Lancet 357:1513-18 (2001). The model has the drawback, however, in that Nair treatment, followed by shaving irritates the skin, thus exacerbating any sensitivity to the applied formulations. Cream formulation vehicle resulted in severe irritation in the Hartley guinea pig model making it impossible to interpret results of docosanol cream treatment to the appropriate vehicle. This irritation does not occur in the hairless model.

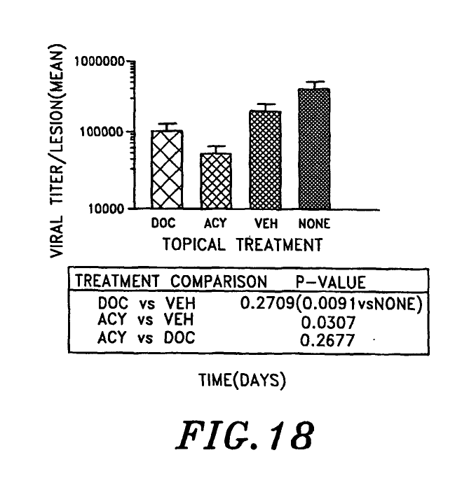

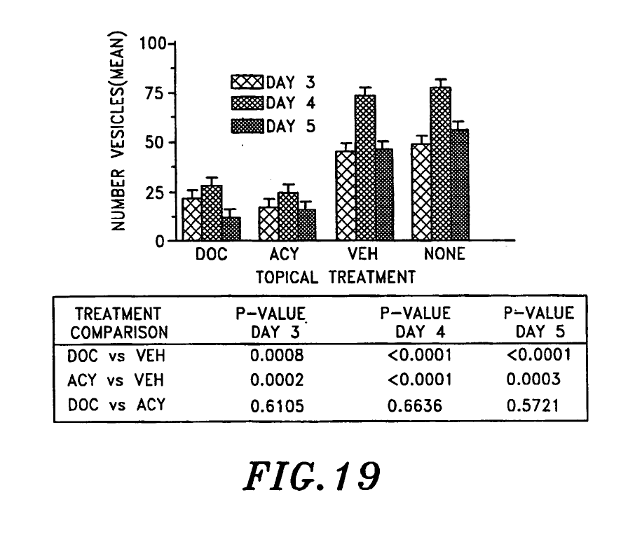

Inoculation of HSV-2 with tattoo gun results in the development of discrete lesions that evolve over time up until Day 6 with complete resolution by Day 9. In the treatment studies, animals were sacrificed on Day 6 for determination of peak viral titer levels. Vesicle numbers were recorded up until the time of sacrifice. Treatment began 12 hours after inoculation and was repeated four times per day on Day 1 through Day 3 and three times per day on Day 4 and Day 5 for a total of 19 treatments. Skin samples were collected on Day 6 for analysis of virus titers. Mean viral titer per lesion is shown in FIG. 18 for each treatment. Statistical information is summarized in the table below the figure. Vesicle numbers observed on Day 3 through Day 5 are shown in FIG. 19, which produced the same pattern of results as lesion viral titers.

Hartley guinea pigs were inoculated with the MS strain of HSV-2 (60 μl 2.9×10 PFU/ml) utilizing a tattoo gun as described in Materials and Methods on each of 6 sites on the dorsal surface. Treatments started twelve hours post inoculation and were repeated 4 times/day for 3 days and three times a day for two days. Vesicle numbers were counted on Days 3, 4, and 5. Lesion skin was collected on Day 6, at 12 hours after the last treatment and assayed for viral content. Each treatment was applied to 9 sites.

Previous studies with docosanol had not included viral titer measurements, but rather focused on decreased disease duration as assessed by counting the number of vesicles until healing occurred. The faster healing times observed in the earlier studies could have resulted from mechanisms of actions not related to an antiviral activity. The results from this study established that docosanol-containing formulations result in decreased viral content in the skin of guinea pigs infected with HSV-1 and HSV-2 in both the hairless (compared to vehicle or untreated sites) and Hartley guinea pig models (compared to untreated sites). The inhibition observed is approximately equivalent to that observed with acyclovir and statistically significant differences between the two treatments were not generally observed.

The anti-HSV activity of a topically applied compound is highly dependent upon the topical vehicle used. See Sidwell et al., “Effect of vidarabine in DMSO vehicle on type 1 herpes virus-induced cutaneous lesions in laboratory animals,” Chemother. 33:141-50 (1987). It appears that both PEG and cream vehicle worked relatively well for delivery of docosanol and acyclovir, although other vehicles could potentially enhance the antiviral activity.

The differing results in the two models may be a result of the irritation induced in the chemically depilated and shaved skin. Irritation induces inflammation that may alter the healing rate. Decreased viral titer levels were observed following docosanol treatment in hairless and Hartley guinea pigs, but statistical significance compared to vehicle treated sites was more reproducibly demonstrated in the hairless model. Decreased viral titer per lesion also correlated with decreased vesicle numbers although the magnitude of the effect was less when vesicle numbers were evaluated.

The hairless guinea pig model with inoculation with a tattoo gun provided reproducible evidence of effectiveness of docosanol formulations in the treatment of cutaneously induced herpes lesions and provides more reproducible results than the Hartley guinea pig model. The results of this study establish that docosanol inhibits replication of HSV in these model systems to an extent approximately equivalent to that of acyclovir ointment, suggesting that its efficacy in the treatment of cold sores may result from its antiviral activity.

n-Docosanol formulated as n-docosanol 10% cream was studied for the topical treatment of herpes simplex infectious. Efficacy in reducing the healing time of recurrent oral-facial herpes simplex infections has been demonstrated in Phase II and in Phase III placebo controlled clinical trials. Positive results were also obtained in a Phase III pilot study using n-docosanol 10% cream as a topical treatment for cutaneous Kaposi's sarcoma lesions in HIV-I positive patients. n-Docosanol topical cream prevented vaginal transmission of SlVmac25I in rhesus macaques, suggesting that the compound has antimicrobial functions that may be useful as a prophylactic to prevent the transmission of HIV in humans.

n-Docosanol exhibits antiviral activity in vitro against a wide range of lipid-enveloped viruses. Susceptible human viruses include HSV-1 and HSV-2 (including acyclovir-resistant strains and clinical isolates), influenza A, respiratory syncytial virus, cytomegalovirus, varicella zoster virus, human herpes virus 6 and HIV-1. The ID₅₀ values (concentration where 50% inhibition is observed) ranged from 3 to 12 mM for these susceptible viruses. Non-enveloped viruses and enveloped viruses that are endocytosed have an apparent resistance to the effects of n-docosanol. For in vitro efficacy studies, the insoluble n-docosanol is formulated by suspending the molecule in the inert and non-toxic surfactant Pluronic F-68, a block copolymer of polyethylene oxide and polypropylene oxide, or a related molecule, Tetronic 908. The relatively high concentrations of n-docosanol required for in vitro activity may be a result of the physiochemical nature of the surfactant-stabilized particles. However, because n-docosanol concentrations as high as 300 mM are not cytotoxic, the therapeutic index for the drug is favorable.

Studies, generally conducted with HSV, demonstrated that n-docosanol does not directly inactivate virus since virus preparations can be mixed with the compound without loss of infectivity. Instead, the drug apparently modifies the target cell in a manner that inhibits viral replication. Studies have demonstrated that radiolabeled n-docosanol is extensively incorporated into host cells and metabolized to phospholipids with the chromatographic properties of phosphatidylcholine and phosphatidylethanolamine. Furthermore, conditions that increase the amount of n-docosanol metabolism increase the amount of antiviral activity, suggesting that this intracellular metabolic conversion of the drug is required for antiviral activity.

n-Docosanol inhibits HSV-induced plaque formation and production of viral particles as judged in a secondary plaque assay. It also inhibits, as determined by ELISA, the production of HSV core and envelope proteins and the number of cells expressing the intranuclear HSV-I specific immediate-early protein. These observations suggested that n-docosanol interferes with an early step in HSV infection.

Studies were conducted to investigate the mechanism of action for the anti-HSV activity of n-docosanol utilizing (1) an HSV recombinant virus which expresses β-galatotsidase on entry of the viral genome into the nucleus of a susceptible host cell; (2) a host cell transformed to express β-galactosidase upon entry of HSV virion proteins into the cell; and (3) HSV-2 fluorescently labeled with octadecyl rhodamine B chloride.

n-Docosanol (98% pure; M. Michel, New York) was suspended in Tetronic 908 (poloxamine 908, Mw 25000; BASF; Parsippany, N.J.) generally as follows. Tetronic 908 was diluted to 1.6 mM in 37° C. sterile saline, and the solution was then heated to 50° C. n-Docosanol was added to 300 mM to the Tetronic in saline and the mixture was sonicated (Branson 450 sonifier; Danbury, Conn.) for 21 min at an initial output of 65 W; this warms the mixture to 86° C. The resulting suspension consists of very fine globular particles with au average size of 0.1 microns as measured by transmission electron microscopy.

Heparin and NP-40 were obtained from Sigma (St. Louis, Mo.) and octadecyl rhodamine B from Molecular Probes (Eugene, Oreg.). Anti-gD neutralizing monoclonal antibody (III-174) was generated. Plaque reduction assays were typically performed in Vero cells (African Green monkey kidney; ATCC no. CCL-81). The HEp-2 (human epidermoid carcinoma; ATCC no. CCL-23), cell line and NC-37 human B cells (ATCC No. CCL214) were obtained from the American Type Culture Collection. The CHO-IEβ8 cell line was developed. It was selected by transfection of Chinese hamster ovary cells (CHO-KI; ATCC no. CCL-61) with a plasmid carrying a puromycin (Pur) selectable marker and lacZ under control of the HSV-I ICP4 promoter. The cell line was selected in Pur and screened for expression of β-galactosidase after HSV infection but not in the absence of infection.

The MacIntyre strain of HSV-I (VR-539) and the MS strain of HSV-2 (VR-540) were obtained from the American Type Culture Collection. HSV-2 (333), a wild-type strain, was obtained from Dr Fred Rapp. Stock preparations were titered for levels of plaque-forming units (PFU) in Vero cells and stored frozen at −80° C. HSV-I(KOS)gL86 is a replicabon-defective mutant in which the gL ORF is replaced with lacZ under control of the CMV promoter. This mutant is propagated in gL-expressing Vero cells and is fully infectious but can undergo only one round of replication in non-complementing cells.

Cultured cells were placed in 35-mm wells (2 ml; 3×10⁵ cells/ml) in DMEM containing L-glucamine, pennstrep (cDMEM) and supplemented with 5% FCS. n-Docosanol or the corresponding control vehicle (lacking n-docosanol) was added at the outset of the culture. All cultures were then inoculated with 175 p.f.u. of HSV-1 or HSV-2.

The cultures were incubated for an additional 42-44 h, washed once with fresh medium, stained and fixed (the staining/fixative consists of 1.25 mg/ml of carbol-fuchsin plus 2.5 mg/ml of methylene blue in methanol) and then scored for HSV-induced plaques using a dissecting microscope (10× magnification). Thc data are averages of duplicate cultures, which varied by no more than 5-10%.

Twenty-four hours before infection, cultured cells were seeded into 24-well (16-mm) plates at 2.5×10⁶ cells/well in 0.5 ml cDMEM supplemented with 10% fetal bovine serum (FES). After cell attachment (4-6 h later) heparin, n-docosanol-surfactant, or surfactant alone was added to the cells in 0.5 ml DMEM/10% FES. The agents were dissolved in the medium at two times the desired final concentration. For infection, 0.7 ml of medium was removed from each well and 25 μl of virus suspension was added to the remaining 0.3 ml to give a virus dose of at least 20 p.f.u./cell The plates were rocked at 37° C. for 3 h and then put in a 37° C. CO₂ incubator for another 2-3 h.

At 5-6 h after infection, the cells were fixed with PES containing 2% formaldehyde and 0.2% glutaraldehyde, washed, then permeabilized with 0.02% NP-40, 0.01% deoxycholate and 2 mM MgCl₂. After washing again, 5-bromo-4-chloro-3indolyl-β-galactopyranoside (X-gal) was added for development of blue product. The substrate was removed and replaced with 50% glycerol. Plates were photographed. To quantify the amount of color in each well the glycerol was removed from all wells that were then washed 3× with distilled H₂O. DMSO (0.6 ml) was added to solubilize the dye and, after transferring 100 μl of each sample well from the 24-well plate to a 96-well plate, the OD₆₀₀ was recorded using a 96-well plate reader.

The HSV envelope was labeled with octadecyl rhodamine B chloride (R-18). NC-37 human B cells were inoculated at 2.5×10⁵ cells/ml, 25 ml per flask. Cells were incubated overnight at 37° C. with no addition or in the presence of 15 mM n-docosanol or the corresponding concentration of Tetronic 908. Cells were harvested by centrifugation and resuspended to 1×10⁶ cells/ml. Aliquots (0.2 ml in test tubes) were chilled for 20 min at 4° C. before the addition of 100 μl R-18 labeled HSV-2. After 3 h at 4° C., 3 ml media containing n-docosanol or Tetronic 908 at the original concentrations were added and the samples were incubated at 37° C. for various times. Cells were centrifuged at 4° C., washed with saline, centrifuged, and resuspended in 10% formalin in saline (3 ml). The cells were washed with saline and resuspended in PBS containing 10% FCS. Fluorescence intensity was measured by using a fluorescence-activated cell sorter (FACScan; Becton-Dickinson).

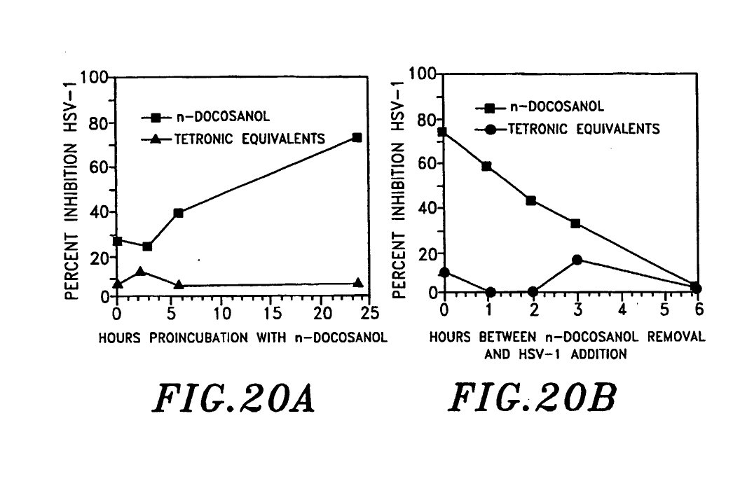

The active form of the drug has a finite lifetime in the cell membrane with a half-life of approximately 3 h. Antiviral activity is increased in target cells incubated with n-docosanol prior to the addition of HSV. This is illustrated in FIG. 20a which shows the effect of incubation time of Vero cells with 9 mM n-docosanol on inhibition of HSV-I-induced plaques. In this experiment, 9 mM n-docosanol inhibited plaque formation in Vero cells 28% when added simultaneously with, or 3 h prior to virus addition; this was increased when cells were treated with drug 6 h prior to inhibition, but the greatest inhibition occurred in cells treated 24 h before HSV-I addition. Intermediate time intervals were not examined.

To establish the length of time Vero cells remain resistant to HSV infection after optimal time of incubation with n-docosanol, Vero cells were incubated with 9 mM n-docosanol for 21-27 h. Media containing unincorporpted drug was then removed and replaced with fresh media. Drug was not replaced. HSV-1 was added immediately, or following a 1-, 3-, or 6-h period of incubation at 37√ C. Two hours following addition of HSV-1, excess virus was removed and the plaque reduction assay was continued as described above. As shown in FIG. 20b, the antiviral activity observed (% inhibition plaque formation) decreased gradually as the time between drug removal and viral addition increased. With a 3-h interval between drug removal and HSV-I addition, 50% of the inhibitory activity was lost; with a 6-h interval no inhibition of HSV-I plaque formation was observed.

Attachment of HSV-I to specific cell surface receptors is unaffected in n-docosanol-treated cells. Previous studies have verified that attachment of HSV-1 to specific cell surface receptors is unaffected in n-docosanol-treated cells. Vero cells incubated with 15 mM n-docosanol bound normal levels of [³H]HSV-I added at 2 p.f.u./cell. Heparin inhibited this interaction 96%. The specificity of the binding assay was confirmed using mouse HSV-I immune sera which reduced binding by 96% compared to normal mouse sera which did not inhibit [³H]HSV binding.

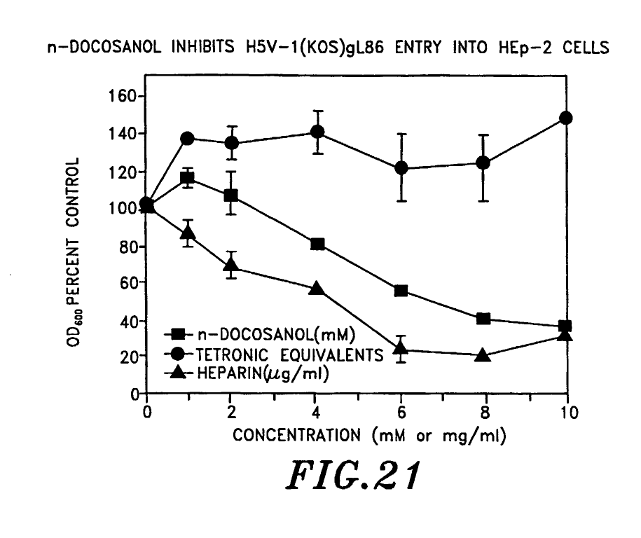

Production of β-gal is inhibited in n-docosanol-treated HEp-2 cells infected with HSV-I(KOS)gL86. To investigate the effects of n-docosanol treatment on entry of HSV into target cells, a viral construct HSV-1(KOS)gL86 was utilized. In this replication-defective mutant, in which lacZ expression is under control of the CMV promoter, β-galactosidase is expressed after entry of the viral genome into a susceptible host cell nucleus. Addition of X-gal results in the development of blue color proportional to the number of cells infected. The intensity of the signal is inhibited by agents such as heparin which block viral binding (see FIG. 21) or agents which prevent entry including neutralizing monoclonal antibodies to gD, an HSV-specific protein required for entry. This signal is not inhibited by acyclovir or other agents that inhibit DNA replication.

The effect of n-docosanol treatment of HEp-2 cells at doses ranging from 0.9 to 9.9 mM (0.333.3 mg/ml) on the entry of HSV-1(KOS)gL86 was examined. HEp-2 cells were incubated for 24 h with the indicated concentrations of n-docosanol suspended in Tetronic 908 prior to addition of the mutant virus. At 5-6 h after infection the cells were fixed and permeabilized and X-gal was added. n-Docosanol treatment resulted in the visibly apparent production of fewer blue cells at n-docosanol concentrations as low as 4 mM. Almost no color development occurred in cells treated with 8 and 10 mM n-docosanol, respectively. To quantify the inhibition of viral infectivity, the substrate within the HEp-2 cells was solubilized by the addition of DMSO and the OD₆₀₀ was recorded as shown in FIG. 21. The ID₅₀ for n-docosanol was approximately 7 mM, roughly equivalent to the ID₅₀ values, 4 and 9 mM, for inhibition of HSV production and plaque formation, respectively, in Vero cells. The vehicle, Tetronic 908, without n-docosanol, was not inhibitory to viral entry. In fact, treatment of cells with equivalent volumes of vehicle enhanced the blue color development as much as 40%. Heparin was examined at concentrations between 1 and 10 μg/ml; inhibition appeared to be complete at 6 μg/ml. These results established that the HSV genome does not effectively enter the nucleus in n-docosanol-treated cells. Combined with the failure of n-docosanol to inhibit viral attachment, this experiment indicate that a step of viral [symbol] is blocked by n-docosanol treatment and that this event occurs subsequent to viral attachment but prior to nuclear entry of the viral genome.

n-Docosanol inhibits HSV-2(333) infectivity of CHO-IEβ8 cells. To further narrow the point of inhibition of viral entry in n-docosanol-treated cells, the effects of the drug on entry of HSV-2 into CHO-IEβ8 cells selected by transfection of CHO cells with a plasmid carrying a Pur selectable marker and lacZ under control of the HSV-1 ICP4 promoter was investigated. In this cell line, β-gal expression is induced upon entry of HSV virion proteins into the cell, an event which occurs immediately upon viral entry into the cellular cytoplasm and which is not dependent on virion transport to the nucleus. Color development is proportional to the number of cells infected and, as in the previous assay, is effectively inhibited by agents such as heparin which block viral attachment and by agents which inhibit entry (such as antibodies to gD) but not by acyclovir and other inhibitors of DNA replication.

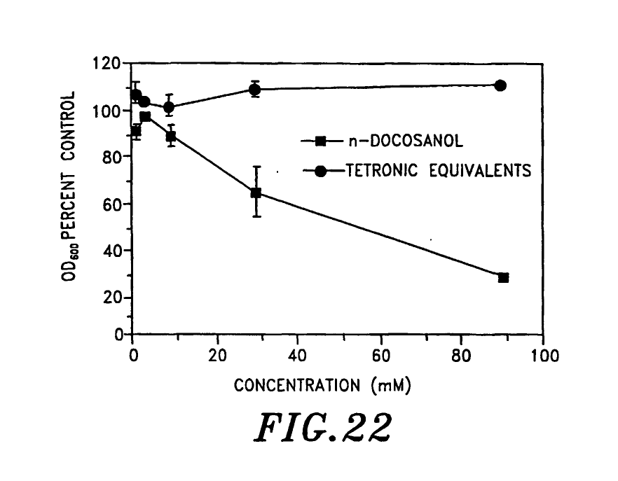

As illustrated in FIG. 22, n-docosanol inhibited β-galactosidase expression in this assay. Whereas treatment of CHO-IEβ8 cells with vehicle alone resulted in a slight increase in OD₆₀₀ (˜10%), n-docosanol treatment of cells results in a concentration-dependent decrease in the color development signifying infected cells. In this experiment 30 mM n-docosanol inhibited color production 40% compared to untreated cells and 55% compared to Tetronic 908-treated cells. The maximal observed inhibition in comparison to untreated cells was approximately 75%. This, in combination with the lack of inhibition in the binding assay, narrows a point of inhibition to an event after viral attachment but prior to release of virion proteins and manifestation of VP16 transactivator activity (an immediate post-entry event not dependent on virion transport to the nucleus).

n-Docosanol-treated NC-37 human B-cells exhibit decreased fusion with octadecyl rhodamine B chloride-labeled HSV-2. Because of the selectivity of the inhibitory effects of n-docosanol for lipid-enveloped fusion-dependent viruses and the absence of viricidal effects, we considered the possibility that n-docosanol may inhibit viral entry by altering target cell membranes to prevent effective fusion of viral particles with target cells. To investigate the effects of n-docosanol on HSV fusion with cellular membranes we conducted fluorescence dequenching assays. The membranes of intact HSV-2 virions were labeled with octadecyl rhodamine chloride (R-18) and added to human B cells. In this model, if viral fusion with the cellular membrane occurs, the tightly packed rhodamine molecules diffuse into the larger membrane of the host cell. This relieves fluorescence self-quenching and causes an increase in signal intensity.

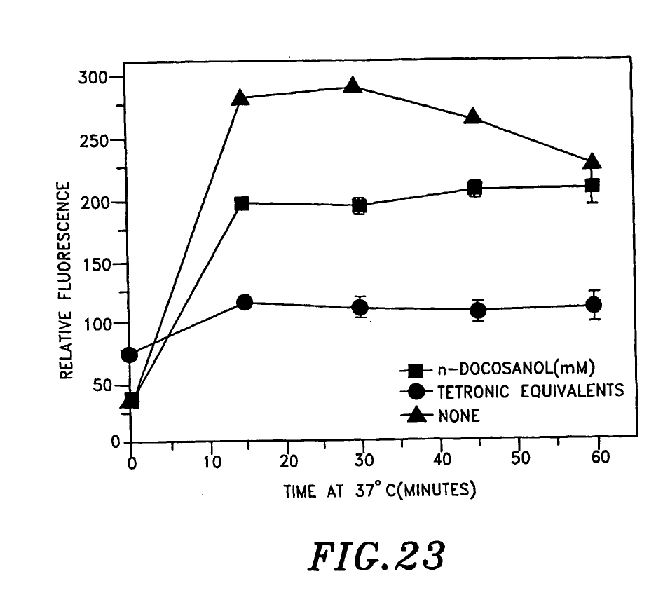

NC-37 human B cells were treated with 15 mM n-docosanol 24 h before the addition of R-18 labeled HSV-2. As shown in FIG. 23, this concentration of n-docosanol inhibited the relative increase in fluorescence intensity occurring with viral/cell fusion by approximately 50% compared to cells receiving no treatment. Treatment of NC-37 cells with Tetronic control suspensions was not inhibitory, and instead caused a noticeable increase in fluorescence intensity, reminiscent of the observation made with the β-gal expressing systems discussed above (FIGS. 21 and 22). Compared to the effect observed with the Tetronic control alone, n-docosanol inhibited the fluorescent response by as much as 76%. n-Docosanol was not inhibitory if added only during the fusion process; a prior incubation period of the compound with cells was necessary. This is consistent with the requirement for metabolic conversion in the antiviral process. The observation also establishes that the presence of n-docosanol does not itself quench or otherwise inhibit fluorescence. Anti-gD monoclonal antibody (a specific inhibitor of penetration) at a 1:40 dilution completely blocked the increase in fluorescence signal (not shown) confirming that the experimental protocol is an appropriate measure of viral penetration. These results indicate that fusion of HSV viral particles to the host membranes is significantly inhibited in n-docosanol-treated cells.

Most available antiviral therapeutic compounds block replication processes shared by the virus and infected target cell and hence are toxic, mutagenic, and/or teratogenic and can potentially induce drug-resistant viral mutant substrains. Therefore, the identification of new antiviral compounds, particularly those with new mechanisms of action, is important. The 22-carbon, saturated, primary alcohol, n-docosanol, lacks any toxic, mutagenic, or teratogenic properties. In contrast to the mode of action of conventional antiviral agents, the predominant mechanism for the anti-HSV activity of n-docosanol appears to be inhibition of fusion between the plasma membrane and the HSV envelope and, as a result, the blocking of entry and subsequent viral replication. The mechanism of action explains the effectiveness of n-docosanol against all tested lipid-enveloped viruses that employ fusion as the sole or major means of entry into the cell and contrasts its mode of action to other antiviral agents that target a single viral protein. Based on this mechanism of action the emergence of HSV strains resistant to the antiviral effects of n-docosanol may be unlikely.

Previous results had suggested that n-docosanol may be specific for lipid-enveloped-viruses, and that lipid-enveloped viruses which primarily enter cell by fusion with the plasma membrane are effectively blocked by n-docosanol. In contrast, the drug generally exerts no detectable activity against viruses that are either non-enveloped, or are enveloped and endocytosed. One exception to this general pattern is influenza A, an enveloped virus that has been reported to enter cells via receptor-mediated endocytosis but which is effectively inhibited by n-docosanol. The reasons for this anomaly are currently unclear.

The in vitro doses (mM) required for antiviral inhibition with n-docosanol are high compared to results with existing therapeutic compounds such as acyclovir. This may result from the nature of the surfactant-stabilized suspensions of n-docosanol. Due to the insolubility of n-docosanol, the particles are thermodynamically stable, making transfer to cultured cells an inefficient process. As determined using radiolabeled n-docosanol, less than 1 out of 1000 molecules of n-docosanol added to culture enters the cell.

Optimal inhibition of viral replication was observed in Vero cell cultures to which HSV was added 6-24 h after addition of n-docosanol. This observation can be explained by a time-dependent uptake and metabolism of n-docosanol by host cells, an event apparently required for antiviral activity. The rate of this metabolic conversion in vivo is likely to be faster than that observed in the artificial milieu of the tissue culture system, especially considering the thermodynamic stability of the surfactant-stabilized particles. The gradual loss of resistance to HSV in n-docosanol-treated cells reported herein would also be predicted due to rapid turnover not only of a required lipid metabolite but of the plasma membrane itself which is constantly being internalized and replaced. However, even with this rapid turnover, viral entry was reduced for several hours following removal of unincorporated drug.

Furthermore, the topically applied cream remains on the skin surface acting as a constant reservoir of n-docosanol. Available data demonstrated that n-docosanol exerts an effect on the host cell that inhibits early events in viral replication but does not inhibit the amount of HSV which attaches to cells. The effect of n-docosanol on progressively earlier events in viral entry was therefore examined.

Penetration of HSV-1(KOS)gL86 into HEp-2 cells was inhibited by n-docosanol with a concentration dependence (ID₅₀=7 mM) roughly equivalent to inhibition of HSV-1 or HSV-2 production (ID₅₀=4 mM) or plaque formation (ID₅₀=9 mM) in Vero cells (FIG. 21) confirming that n-docosanol inhibits an early event in the viral replication cycle. The inhibitory activity of n-docosanol on β-galactosidase expression must counteract the apparent stimulatory action of the vehicle alone, the mechanism for which is unclear. n-Docosanol inhibition of HSV-2 entry was also evidenced by reduced release into treated cells of virion-associated regulatory proteins (FIG. 22). n-Docosanol treatment caused as much as an 80% reduction in the expression of β-galactosidase in target cells containing a stably transfected lacZ gene under control of an HSV immediate early promoter (ICP4). This observation, in combination with the lack of inhibition of viral attachment in n-docosanol-treated cells, confirms that n-docosanol blocks an event occurring after viral attachment but prior to release of tegument proteins. This is an immediate post-entry event and is not dependent upon virion localization in the nucleus. The inhibitory concentrations were higher than that generally required for in vitro anti-HSV activity. Additional early events in viral replication may also be inhibited by n-docosanol.

n-Docosanol appears to inhibit the biophysical process of viral/cell fusion. The fusion-dependent dequenching of octadecyl rhodamine B chloride, inserted into the HSV envelope was significantly inhibited in n-docosanol-treated cells (FIG. 23). The concentration dependence of fluorescence inhibition correlated to that observed for inhibition of HSV-1 replication by n-docosanol in other in vitro assays. Incorporation of n-docosanol, or its metabolites, and resulting perturbations of normal membrane composition may alter the biophysical properties of the plasma membrane in such a way as to inhibit fusion of attached virions. The compound may inhibit the function of normally occurring cellular mediators of entry.

Inhibition of fusion between the plasma membrane and the HSV envelope, and the subsequent lack of replicative events, may be the predominant mechanism for the anti-HSV activity of n-docosanol. This mechanism of action may be generally applicable to the spectrum of viruses susceptible to the inhibitory effect of n-docosanol.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. All patents, applications, and other references cited herein, are hereby incorporated by reference in their entirety.

What is claimed is:

1. A therapeutic cream for application to skin and mucous membranes in the treatment of viral and inflammatory diseases consisting essentially of about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water.

2. A method of treating viral infections and inflammations of skin and mucous membranes comprising applying to the skin or mucous membranes a stable therapeutic topical cream consisting essentially of about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water.

3. A method of reducing the pain of a surface inflammation of skin and mucous membranes comprising applying to the inflamed surface a composition consisting essentially of about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water.

4. Use of a composition consisting essentially of about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water, in the preparation of a medicament for treatment of viral infections and inflammation of the skin or mucous membranes.

5. Use of a composition consisting essentially of about 10 wt. % n-docosanol; about 5 wt. % of a stearate selected from the group consisting of sucrose monostearate, sucrose distearate, and mixtures thereof; about 8 wt. % light mineral oil; about 5 wt. % propylene glycol; about 2.7 wt. % benzyl alcohol; and about 69.3 wt. % water, in the preparation of a medicament for reducing the pain of a surface inflammation of the skin or mucous membranes.

6. A therapeutic cream for application to skin and membranes in the treatment of viral and inflammatory diseases consisting essentially of sugar-based ester surfactant, greater than about 5 wt. % n-docosanol, mineral oil, an emollient co-solvent, and water.

7. The therapeutic cream of claim 6 wherein the cream is stable at temperatures of at least 40° C. for a period of at least three months and after repeated freeze-thaw cycles.

8. The therapeutic cream of claim 6 wherein the sugar-based ester surfactant is selected from the group consisting of sucrose cocoate, sucrose stearates and sucrose distearate.

9. The therapeutic cream of claim 8 wherein the sugar-based ester surfactant comprises at least one compound selected from the group of sucrose esters consisting of sucrose cocoate, sucrose stearates and sucrose distearate, wherein sucrose ester (s) comprise about 3 wt. % or more of the cream.

10. The therapeutic cream of claim 9 wherein sucrose ester(s) comprise about 5 wt. % or more of the cream.

11. The therapeutic cream of claim 6 wherein the emollient co-solvent is selected from the group consisting of polyoxypropylene stearyl ether, ethyl hexanediol, and benzyl alcohol, or combinations thereof.

12. The therapeutic cream of claim 6 wherein the n-docosanol comprises at least approximately 10 wt. % of the cream.

13. A stable, efficacious therapeutic cream wherein a principal therapeutic composition consists essentially of n-docosanol, and wherein the cream base comprising one or more compounds selected from the group consisting of sucrose cocoate, sucrose stearates and sucrose distearate and one or more compounds selected from the group consisting of polyoxypropylene stearyl ether, ethyl hexanediol, and benzyl alcohol.

14. The therapeutic cream of claim 13 wherein sucrose ester(s) comprise at least approximately 5 wt. % of the cream.

15. The therapeutic cream of claim 13 wherein the n-docosanol comprises at least approximately 10 wt. % of the cream.

16. The therapeutic cream of claim 13 having the formulation: n-docosanol comprising from 5 to 15 wt. % of the total cream; sucrose stearates comprising from 0 to 15 wt. % of the total cream; sucrose cocoate comprising from 0 to 10 wt. % of the total cream; sucrose distearate comprising from 0 to 10 wt. % of the total cream; with the proviso that at least one sucrose ester be present and comprise at least about 3 wt. % of the total composition; mineral oil comprising from 3 to 15 wt. % of the total cream; benzyl alcohol comprising from 0.5 to 10 wt. % of the total cream; and water comprising from 40 to 70 wt. % of the total cream.

17. A method of treating viral infections and inflammations of skin and mucous membranes comprising applying a stable therapeutic topical cream wherein the therapeutically active composition consists essentially of n-docosanol, and wherein the cream base consists essentially of sugar-based ester surfactant, at least one long chain aliphatic alcohol having from 20 to 28 carbon atoms selected from the group consisting of n-icosanol, n-henicosanol, n-tricosanol, n-tetracosanol, n-pentacosanol, n-hexacosenol, n-heptacosanol, and n-octacosanol, or mixtures thereof, mineral oil, an emollient co-solvent, and water.

18. The method of claim 17 wherein n-docosanol comprises more than one-half of the long chain aliphatic alcohols

19. A method of treating viral infections and inflammations of skin and mucous membranes comprising applying a topical cream having the formulation:
n-docosanol about 5-20 wt. %;
sucrose stearates about 0-15 wt. %;
sucrose cocoate about 0-10 wt. %;
sucrose distearate about 0-10 wt. %, with the proviso that at least one sucrose ester be present and, wherein sucrose ester(s) comprise about 3 wt. % or more of the cream;
mineral oil about 3-15 wt. %;
propylene glycol about 2-10 wt. %;
polyoxypropylene-15 stearyl ether about 0-5 wt. %;
benzyl alcohol about 0.5-5 wt. %;
with the proviso that either polyoxypropylene stearyl ether or benzyl alcohol be present in an amount of at least about 1 wt. %; and
water about 40-70 wt. %.

20. The method of claim 19 wherein sucrose ester(s) comprise about 5 wt. % or more of the cream.

21. An anti-inflammatory and antiviral cream having the formulation:
n-docosanol about 5-20 wt. %;
sucrose stearates about 0-15 wt. %;
sucrose cocoate about 0-10 wt. %;
sucrose distearate about 0-10 wt. %, with the proviso that at least one sucrose ester be present and wherein sucrose ester (s) comprise about 3 wt. % or more of the cream;
mineral oil about 3-15 wt. %;
propylene glycol about 2-10 wt. %;
polyoxypropylene stearyl ether about 0-5 wt. %;
benzyl alcohol about 0-5 wt. %;
with the proviso that either polyoxypropylene stearyl ether or benzyl alcohol be present in an amount of about 1 wt. % or more; and
water about 40-70 wt. %.

22. The anti-inflammatory cream of claim 21 wherein sucrose ester(s) comprise about 5 wt. % or more of the cream.

23. A method of reducing the pain of a surface inflammation of the skin or membrane comprising applying to the inflamed surface a composition of n-docosanol in a physiologically compatible carrier, said n-docosanol comprising from about 5 to about 25 wt. % of said composition.

24. The method of claim 23 wherein the physiologically compatible carrier is a cream base that comprises one or more compounds selected from the group consisting of sucrose cocoate, sucrose stearates and sucrose distearate and one or more compounds selected from the group consisting of polyoxypropylene stearyl ether, ethyl hexanediol, and benzyl alcohol.
